(19) United States
(12) Patent Application Publication
(10) Pub. No.: US 2013/0252897 A1
(43) Pub. Date: Sep. 26, 2013

(54) Methods and Compositions Regarding Polychalcogenide Compositions

(71) Applicants: Mark B. Roth, Seattle, WA (US); Mike Morrison, Seattle, WA (US)

(72) Inventors: Mark B. Roth, Seattle, WA (US); Mike Morrison, Seattle, WA (US)

(73) Assignee: FRED HUTCHINSON CANCER RESEARCH CENTER, Seattle, WA (US)

(21) Appl. No.: 13/619,933

(22) Filed: Sep. 14, 2012

Related U.S. Application Data
(63) Continuation of application No. 12/146,354, filed on Jun. 25, 2008, now abandoned.
(60) Provisional application No. 60/946,065, filed on Jun. 25, 2007.

(51) Int. Cl. C07K 17/14

(52) U.S. Cl. CPC C07K 17/14
USPC 514/15.2; 530/408; 530/362

(57) ABSTRACT

The present invention concerns the use of polychalcogenide compositions on cells, tissue, organs, and organisms to enhance their survivability. It includes compositions, compounds, methods, articles of manufacture and apparatuses for enhancing survivability and for protecting them from or treating them for injury or damage. In specific embodiments, there are also therapeutic methods and apparatuses for hypoxic/ischemic injury, organ transplantation, hyperthermia, wound healing, hemorrhagic shock, cardioplegia for bypass surgery, neurodegeneration, hypothermia, and cancer using the polychalcogenide compositions described.

This application is a continuation of U.S. patent application Ser. No. 12/146,354 filed on Jun. 25, 2008, which claims priority to U.S. Provisional Patent Application Ser. No. 60/946,065, filed Jun. 25, 2007, which is incorporated herein by reference.

This invention was made with government support under grant number GM048435 awarded by the National Institute of General Medical Sciences (NIGMS). The government has certain rights in the invention.

### BACKGROUND OF THE INVENTION

1. Field of the Invention

The present invention relates generally to the fields of cell biology, physiology, and medicine. More particularly, it concerns methods, compositions and apparatuses for enhancing survivability of and/or reducing damage to cells, tissues, organs, and organisms using one or more polychalcogenide formulations. In certain embodiments, the present invention includes methods, compositions and apparatuses for treating and preventing, and diagnosing diseases and conditions by providing an effective amount of a polychalcogenide formulation to a subject.

2. Description of Related Art

A number of the leading causes of death in the United States involve ischemic or hypoxic damage to cells within an organism. Such causes include heart disease, stroke, bleeding to death, and other related injuries. Every year, hundreds of thousands of people die because of the damage they incur when cells, tissues, and organs do not receive the oxygen they need from blood as a result of these diseases and conditions.

Moreover, while treatment may be available to subjects to prevent the ensuing damage, time remains a significant factor in whether treatment will work. There remains a need for preventative and therapeutic regimens that can provide protection to biological matter in case it is subjected to adverse conditions that may lead to subsequent damage.

### SUMMARY OF THE INVENTION

The present invention addresses the needs discussed above by providing compounds, compositions and methods that enhance the survivability of biological matter and/or protect biological matter from injury or damage. The present invention is based on data showing, for example, a polychalcogenide composition that prevented a subject from succumbing to the adverse effects of hemorrhagic shock. Moreover, the composition exhibited an ability to stave off the effects of tachycardia without substantially altering the subject's mean blood pressure.

As such, the present invention provides methods, compositions, articles of manufacture, and apparatuses involving a polychalcogenide composition to prevent, treat, or enhance the chances of surviving injury or damage to cells, tissues, or organs. In addition, aspects of the invention may reduce the risks and negative outcomes from, for example, ischemic/hypoxic injury, including death.

It is further contemplated that the present invention can be used to enhance survivability of and/or to prevent, protect, treat, or reduce damage (e.g., trauma or injury) to biological matter, such as a cell, tissue, organ or an organism (e.g., a subject), which may or may not be subject to or under adverse conditions. Such methods may comprise providing to the biological matter an effective amount of a polychalcogenide composition. In some embodiments of the invention, increasing survivability refers generally to living longer, which is an embodiment of the invention.

It will be understood that the preventing damage or injury to biological matter does not necessarily mean the biological matter is not damaged or injured at all; instead, typically, the damage or injury is reduced as a result of the polychalcogenide composition relative to the damage or injury that would result in its absence.

As used herein, a “polychalcogenide composition” comprises (i) a peptide or a protein and (ii) a polychalcogenide salt. Typically, the peptide or the protein is in solution, such as an aqueous solution. The composition may be formulated such that the peptide or the protein is stable, such as by adjusting pH and/or ionic strength of a solution in which the peptide or protein is placed. Methods of preparing stable peptide and stable protein solutions are well-known in the art. A “polychalcogenide salt” is a compound of 2 or more consecutive chalcogens, wherein each chalcogen may be the same or different, wherein at least one of the chalcogens is in ionic form, and having at least one counterion. In certain embodiments, a polychalcogenide salt has only one counterion. For example, the following compounds are contemplated as polychalcogenide salts:

ZSₙRₐ (VII)

ZSeₘRₐ (VIII)

ZSₙSeₘRₐ (IX)

wherein Z is a counterion, Rₐ is alkyl, and n and m are as described herein. In certain embodiments, a polychalcogenide salt may be further defined as any one of the following compounds:

ZSₙM (X)

ZSeₘM (XI)

ZSₙSeₘM (XII)

wherein Z is a counterion, M is a counterion, hydrogen or alkyl and n and m are as defined herein. Polychalcogenide salts are well-known in the art, and are also discussed herein (see, e.g., compounds (I), (II) and (III) below). Non-limiting examples of polychalcogenide salts include Na₂S₂, Na₂S₃, Na₂S₄, Na₂S₆, Na₂Se₂, Na₂Se₃, Na₂Se₄, Na₂Se₆, ZnS_{0.5}Se_{0.5}, In₂Te₅, sodium methyl polysulfide, sodium ethyl polysulfide, sodium propyl polysulfide, sodium allyl polysulfide, sodium sulfotrithioite, and potassium ion and cesium ion counterparts of the sodium-containing species.

Chalcogens are members of Group 6 of the periodic table. These elements are oxygen (O), sulfur (S), selenium (Se), tellurium (Te) and polonium (Po). Common chalcogens are S, Se and Te. Chalcogens may be found in elemental forms, such as micronized and/or nanomilled particles of S and/or Se. The present invention also contemplates chalcogens comprising all isotopes of the known chalcogens, including radioisotopes (e.g., ³⁵S, ⁷⁵Se).

In certain embodiments, a polychalcogenide composition is contemplated comprising (i) a peptide or a protein and (ii) a compound of formula (I), (II), and/or (III):

Z₂Sₙ (I)

Z₂Seₘ (II)

Z₂SₙSeₘ (III)

wherein Z is a counterion and n and m are each an integer or non-integer ranging from 1-35, or n and/or m is an average number ranging from 1-35 (including integers and non-integers), or, in the case of compound (III), n+m=1-35 (including integers and non-integers) or an average number ranging from 1-35 (including integers and non-integers). Such compositions may be employed in any embodiment (e.g., method; pharmaceutical composition, etc.) described herein, either alone or in combination. In some embodiments, a polychalcogenide composition further comprises a polychalcogenide compound, as this term is defined below, such as a polychalcogenide compound of formula (IV), (V), or (VI), as described below. In this or any embodiment described herein, an integer may be a half-integer (e.g., 0.5, 1.5, 2.5, 3.5, etc.). In certain embodiments, n equals at least or at most 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, or 35, or more, or any range derivable therein. In certain embodiments, n represents an average number of any of these values. In certain embodiments, n is an average number equal to 5.5. In certain embodiments, n=2-35. The peptide and/or the protein may comprise a chalcogen, such as a sulfur. The peptide or the protein may be a glutathione or albumin, respectively. A glutathione may be reduced glutathione (GSH) or oxidized glutathione (GSSG). Albumin may be human serum albumin, for example. A polychalcogenide composition may be an aqueous composition, for example. The present invention contemplates embodiments comprising one or more polychalcogenide compositions. In certain embodiments, the polychalcogenide composition is further defined as (i) a peptide or a protein, such as glutathione or albumin, in combination with (ii) a compound of formula (I). Such a composition may be termed a “polysulfide composition.” Similarly, polyselenide compositions and poly(sulfide/selenide) compositions are also contemplated.

The percent or amount of peptide or protein in a polychalcogenide composition may vary. Moreover, the percent of peptide or protein in a component of a polychalcogenide composition may vary. For example, in any polychalcogenide composition or in any component of a polychalcogenide composition, the percent or amount of peptide or protein may be about, at most about, or at least about 0.001, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 mg, mg/ml, mM, M, wt/wt % or wt/vol %. In certain embodiments, the wt/wt % of human serum albumin in a polychalcogenide composition is 15%. In certain embodiments, the amount of glutathione is 30 mg/ml. In certain embodiments, an aqueous 15% wt/wt human serum albumin solution is combined with a 30 mg/ml aqueous glutathione solution and 2 mg/kg sodium polysulfide oil to produce a polychalcogenide composition (in this case, a polysulfide composition). This composition may comprise a polychalcogenide compound, as described herein.

Moreover, in any embodiment described herein, a polychalcogenide composition, as described above, may comprise a polychalcogenide compound. As used herein, a “polychalcogenide compound” refers to a compound comprising an unbranched, branched, or cyclic ° chain of 2 or more consecutive chalcogens, wherein each chalcogen may be the same or different, and wherein the compound comprises at least one peptide or protein. A polychalcogenide compound comprising two or more types of chalcogens may be termed a multichalcogenide compound. A polychalcogenide compound may be used, either alone or in combination, with any embodiment described herein. For example, a polychalcogenide compound may be provided to biological matter to enhance the survivability of the biological matter.

In stating that a polychalcogenide compound “comprises” a peptide or a protein, it is to be understood that the interaction between the 2 or more chalcogens and the peptide(s) or protein(s) of a polychalcogenide compound is through any interaction known in the art, such as a covalent bond (e.g., polar covalent, non-polar covalent), an ionic bond, or a hydrogen bond (wherein the chalcogen acts as a hydrogen bond acceptor). More than one type of interaction between 2 or more chalcogens and the peptide or protein is also contemplated, meaning that different chalcogens may participate in different types of interactions within the same compound. In certain embodiments, the interaction comprises a disulfide bond, in which a sulfur atom from the peptide or the protein participates.

In some embodiments, a polychalcogenide compound is a polysulfide compound. As used herein, a “polysulfide compound” refers to a polychalcogenide compound comprising an unbranched, branched, or cyclic chain of 2 or more consecutive sulfur atoms, wherein the compound comprises at least one peptide or protein. Compositions comprising a mixture of polysulfide compounds are also contemplated.

In certain embodiments, a polychalcogenide compound is a polyselenide compound. As used herein, a “polyselenide compound” refers to a polychalcogenide compound comprising an unbranched, branched, or cyclic chain of 2 or more consecutive selenium atoms, wherein the compound comprises at least one peptide or protein. Compositions comprising a mixture of polyselenide compounds are also contemplated.

The present invention also contemplates poly(sulfide/selenide) compounds as polychalcogenide compounds. As used herein, a “poly(sulfide/selenide) compound” refers to a compound comprising an unbranched, branched, or cyclic chain of sulfur and selenium atoms in any combination, wherein at least 2 of the sulfur and/or selenium atoms are consecutive, wherein the compound comprises at least one peptide or protein. Compositions comprising a mixtures of these compounds are also contemplated.

In certain embodiments, a polychalcogenide compound is defined as a compound of formula (IV), (V), and/or (VI):

XSₙY (IV)

XSeₘY (V)

XSₙSeₘY (VI)

wherein X and Y are each independently a counterion, hydrogen, alkyl_{(C≦6)} a peptide, or a protein, and n and m are each independently an integer or non-integer ranging from 1-35, or n or m is an average number ranging from 1-35. In certain embodiments, at least one of X or Y is a peptide or a protein. In certain embodiments, the peptide or protein is a glutathione or albumin, respectively. Accordingly, either X or Y, or both, may be a glutathione or albumin. Other peptides and proteins are described herein. In certain embodiments, a compound of formula (IV) is contemplated. A compound of formula (IV) may be considered a type of polysulfide compound. In certain embodiments, the counterion is an alkali metal ion. In certain embodiments, the counterion is Na⁺. n and m may each be any value or range of values as described herein, such as an average value.

Moreover, in any embodiment described herein, a polychalcogenide salt may be employed. In certain embodiments, the polychalcogenide salt is a compound of formula (I), as described above. In certain embodiments, the polychalcogenide salt is a compound of formula (IV), (V), or (VI), as described above, but wherein both X and Y are counterions, which may be the same or different. Thus, a “polychalcogenide salt” is not a “polychalcogenide compound,” as each of those terms are used herein. In particular embodiments regarding polychalcogenide salts of formulas (I)-(VI), n and m may be any value or range of values as described herein for these variables. A polychalcogenide composition may comprise a polychalcogenide compound and/or a polychalcogenide salt. A polychalcogenide salt may be used, either alone or in combination, with any embodiment described herein. For example, a polychalcogenide salt may be provided to biological matter to enhance the survivability of the biological matter.

Also contemplated by the present invention are compounds comprising a Ch-(Ch)ₙ-Ch group, wherein each Ch is an atom that is a chalcogen, each Ch may be the same or different, and each terminal Ch may independently comprise another atom or group or may not, and n is a number or an average number ranging from 1-35, including integers and non-integers, or any other value of n described herein. It is to be noted that when n is 2 or more, the Ch within the parentheses may independently vary; it is merely for simplicity that “(Ch)ₙ” is used. For example, Ch-(Ch)₃-Ch may represent, e.g., any of the following groups: S—S—S—S—S, S—S—Se—S—S, Se—S—Se—S—Se, S—Se—S—S—S, as well as other groups, such as groups comprising other chalcogens. Moreover, a compound comprising a Ch-(Ch)₃-Ch group may be, for example, octyl-S—S—Se—S—S⁻, Na^{+ -}S—Se—Se—Se—S-(1-dodecenyl), or glutathione-S—S—S—S—S-glutathione. The word “comprising” in the phrase, “a compound comprising a Ch-(Ch)ₙ-Ch group” or in the phrase “each terminal Ch may independently comprise another atom or group” is to be understood as explained above regarding “comprising” in the context of when a polychalcogenide compound “comprises” a peptide or a protein. In certain embodiments, a compound comprising a Ch-(Ch)ₙ-Ch group is further defined as a compound that comprises two consecutive sulfur-sulfur bonds.

It is specifically contemplated that a compound comprising a Ch-(Ch)ₙ-Ch group may be employed in any embodiment herein. For example, such a compound may be employed in methods for enhancing the survivability of biological matter and/or protecting biological matter from injury or damage. Such compounds may be comprised in any composition described herein, such as a polychalcogenide composition or a pharmaceutical composition. Further, any method herein may employ a compound comprising a Ch-(Ch)ₙ-Ch group. Compounds and compositions may be combined with a compound comprising a Ch-(Ch)ₙ-Ch group as well. For example, a compound comprising a Ch-(Ch)ₙ-Ch group may be combined with a peptide or a protein to form a polychalcogenide composition. In addition, a compound comprising a Ch-(Ch)ₙ-Ch group may be further defined as a polychalcogenide compound or polychalcogenide salt, in certain embodiments,

A compound comprising a Ch-(Ch)ₙ-Ch group may comprise three or more Ch atoms that are branched, linear, cyclic, or any combination thereof. In certain embodiments, a Ch-(Ch)ₙ-Ch group comprises a Ch that is double bonded to another Ch (e.g., a=Ch group, such as ═S). In certain embodiments, a compound comprising a Ch-(Ch)ₙ-Ch group that is branched comprises a ═S group. In certain embodiments, one or more Ch atoms in a compound comprising a Ch-(Ch)ₙ-Ch group is sulfur or selenium. In certain embodiments, each Ch is sulfur. In certain embodiments, A Ch-(Ch)ₙ-Ch group may further comprise additional atoms or groups, wherein those groups may be compounds, peptides and/or proteins, as the word “comprise” is used above in the context of when a polychalcogenide compound “comprises” a peptide or a protein. For example, a Ch-(Ch)ₙ-Ch group may further comprise at least one peptide or at least one protein. The peptide may be a glutathione. The protein may be albumin. The peptide or the protein may comprise a chalcogen, such as sulfur, or may be any other peptide or protein described herein. The Ch-(Ch)ₙ-Ch group may form a covalent bond, such as a disulfide bond, with a peptide or a protein, for example: this is a particular example of a Ch-(Ch)ₙ-Ch group “comprising” a peptide or a protein. The Ch-(Ch)ₙ-Ch group may form an ionic bond with an atom (e.g., an Na⁺ ion), a peptide or a protein, for example: these are other examples of a Ch-(Ch)ₙ-Ch group “comprising” a peptide or a protein. In certain embodiments, n is 7 or greater. In other embodiments, n is an average number equal to 5.5. The compound comprising a Ch-(Ch)ₙ-Ch group may further comprise an alkyl group. The alkyl group may be a C1-C6 alkyl group (that is, alkyl_{c≦6}). The alkyl group may be covalently bound to the Ch-(Ch)ₙ-Ch group, such as through a thioether bond. A Ch-(Ch)ₙ-Ch group may have an overall net charge, or it may not. In certain embodiments, either or both terminal chalcogens of the Ch-(Ch)ₙ-Ch group are ions. In certain embodiments, a compound comprising a Ch-(Ch)ₙ-Ch group may further comprise at least one counterion. The counterion may be an alkali metal ion, such as Na⁺.

The oxidation state of any chalcogen in a Ch-(Ch)ₙ-Ch group may be any oxidation state known to those in the art for the various chalcogens, and the oxidation state of each Ch atom in the Ch-(Ch)ₙ-Ch group may be the same or different. In certain embodiments, the oxidation state of a chalcogen ranges from −2 to 6. In certain embodiments, the oxidation state of sulfur in a Ch-(Ch)ₙ-Ch group ranges from −2 to 6. In certain embodiments, the oxidation state of any Ch atom in a Ch-(Ch)ₙ-Ch group, such as a sulfur atom, may be about, at most about, or at least about −2, −1.9, −1.8, −1.7, −1.6, −1.5, −1.4, −1.3, −1.2, −1, −0.9, −0.8, −0.7, −0.6, −0.5, −0.4, −0.3, −0.2, −0.1, 0, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9 or 6.

Methods of making a variety of compounds comprising a Ch-(Ch)ₙ-Ch group are known in the art. See, e.g., Devillanova, 2006, Steudel, Top. Curr. Chem., vol. 230, 2003a. Steudel, Top. Curr. Chem., vol. 231, 2003b, Guo and Zhang, 2000, Arisawa and Yamaguchi 2003, and Chivers, 2005, each of which is incorporated herein by reference. Moreover, methods described herein offer other means of making such compounds.

It is contemplated that any compound described herein that falls into the category of a compound comprising a Ch-(Ch)ₙ-Ch group may be specifically excluded from the definition of a compound comprising Ch-(Ch)ₙ-Ch group. Moreover, the present invention further contemplates a compound comprising a Ch-(Ch)ₙ-Ch group, wherein the compound is further defined as not any compound as disclosed in any of the following documents, each of which is incorporated herein by reference in its entirety: U.S. Patent Publns. 2005/0053912; 2005/0170019; 2005/0147692; 2005/0136125; 2008/0085329; or 2007/0078113; or U.S. patent application Ser. Nos. 11/837,536; 11/837,491; 11/837,539; 11/738,294; 11/864,355; 12/016,886; or 60/946,065. In certain embodiments, the present invention contemplates a compound comprising a Ch-(Ch)ₙ-Ch group, wherein the compound is further defined as not any one or more of the following, or any combination thereof, wherein (i) and (ii) are described in, e.g., U.S. Patent Publn. 2007/0078113, incorporated herein by reference in its entirety:

(i) a compound of formula (A):

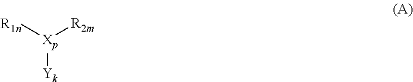

wherein: X is a halogen, Po, S, Se, or Te; R₁ and R₂ are each independently H, lower alkyl, amine, lower amine, a short chain alcohol, a short chain ketone, a C₂-C₅ ester, a C₂-C₅ amide, a C₂-C₅ aldehyde, a C₂-C₅ ketone, a C₂-C₅ carboxylic acid, a C₂-C₅ ether, a C₂-C₅ nitrile, a C₂-C₅ anhydride, a C₂-C₅ halide, a C₂-C₅ acyl halide, a C₂-C₅ sulfide, a C₂-C₅ sulfone, a C₂-C₅ sulfonic acid, a C₂-C₅ sulfoxide, or a C₂-C₅ thiol; a short chain ester, a short chain amide, a short chain aldehyde, a short chain ketone, a short chain carboxylic acid, a short chain ether, a short chain nitrile, a short chain anhydride, a short chain halide, a short chain acyl halide, a short chain sulfide, a short chain sulfone, a short chain sulfonic acid, a short chain sulfoxide, and/or a short chain thiol; n is 1; m is 1; k is 0; and p is 1 or 2, wherein the compound may optionally be a cyclic compound and wherein optionally R₁ and R₂ may be bridged;

(ii) a compound of formula (B):

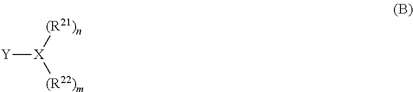

wherein: X is Po, S, Se, Te, Po—Po, S—S, Se—Se, or Te—Te; n and m are independently 0 or 1; R²¹ and R²² are independently hydrogen, alkyl, alkenyl, alkylthio, cycloalkyl, or cycloalkenyl; and Y is —R²³R²⁴, wherein R²³ is S, SS, Po, Po—Po, Se, Se—Se, Te, or Te—Te, and R²⁴ is defined as for R²¹ above, or Y is

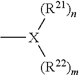

wherein X, R²¹ and R²², are as defined above;
and/or

(iii) wherein if the compound is of any of formulas (XIII), (XIV), or (XV),

XSₙY (XIII)

XSeₘY (XIV)

XSₙSeₘY (XV)

wherein X and Y are each independently hydrogen, alkyl_{(C≦6)}, or alkenyl_{(C≦6)}, and n and m are each independently an integer or non-integer ranging from 1-35, and if three or more S and/or Se atoms in a compound of formula (XIII), (XIV), or (XV) are each comprised as a linear chain of S, Se, or S/Se atoms and if n, m, or n+m is less than or equal to 6, then: X and Y are not both hydrogen; X and Y are not both alkyl_{(C≦6)} or alkenyl_{(C≦6)}; X is not hydrogen when Y is alkyl_{(C≦6)} or alkenyl_{(C≦6)}; and Y is not hydrogen when X is alkyl_{(C≦6)} or alkenyl_{(C≦6)}. “Lower” is meant to refer to 1, 2, 3, 4, 5, or 6 carbon atoms, or any range derivable therein. “Short chain” means 1, 2, 3, 4, 5, 6, 7, 8, 9, 10, 11, or 12 carbon molecules, or any range derivable therein. “Alkylthio” refers to wherein an alkyl radical is substituted with one or more thiol radicals (e.g., —S—CH₃). The term “alkenyl” refers to an unsaturated, acyclic hydrocarbon radical in so much as it contains at least one double bond. Such alkenyl radicals contain from about 2 to about 20 carbon atoms. The term “lower alkenyl” refers to C₁-C₆ alkenyl radicals. As used herein, the term alkenyl radicals includes those radicals substituted as for alkyl radicals. Examples of suitable alkenyl radicals include propenyl, 2-chloropropenyl, buten-1-yl, isobutenyl, pent-1-en-1-yl, 2-2-methyl-1-buten-1-yl, 3-methyl-1-buten-1-yl, hex-2-en-1-yl, 3-hydroxyhex-1-en-1-yl, hept-1-en-1-yl, and oct-1-en-1-yl, and the like.

Moreover, the present invention also contemplates methods comprising providing to biological matter (e.g., a subject) a prodrug, wherein the prodrug is converted upon provision to a compound comprising a Ch-(Ch)ₙ-Ch group. In certain embodiments, a prodrug is provided to biological matter that, upon provision, is oxidized to form a compound comprising a Ch-(Ch)ₙ-Ch group. The prodrug may be a compound comprising a Ch-(Ch)ₙ-Ch group that is converted (e.g., oxidized) to a different compound comprising a Ch-(Ch)ₙ-Ch group. Moreover, a prodrug may be a polychalcogenide composition, a polychalcogenide compound, or a polychalcogenide salt, as those terms are defined herein.

Examples of compounds comprising a Ch-(Ch)ₙ-Ch group can be found throughout this application. Moreover, the following types of compounds are also compounds that comprise a Ch-(Ch)ₙ-Ch group: polysulfane species, polysulfide species, hydropolysulfide species, cyclic polysulfur species, polyselane species, polyselenide species, hydropolyselenide species, cyclic polyselenide species, and poly(sulfur/selenium) species, as each of these terms are defined below.

As used herein, a “polysulfane species” refers to a compound having an unbranched or branched chain of sulfur atoms terminating in H: HSₙH, wherein n is as defined herein, provided n is 2 or more. As used herein, a “polysulfide species” refers to a compound having an unbranched or branched chain of sulfur atoms of the following formula: XSₙY, wherein X and Y are independently not H, and n is as defined herein, provided n is 2 or more. As used herein, a “hydropolysulfide species” refers to a compound having an unbranched or branched chain of sulfur atoms of the following formula: RSₙH, wherein R is not H and n is as defined herein, provided n is 2 or more. As used herein, a “cyclic polysulfur species” comprises a ring of sulfur atoms, wherein the ring may comprise 3 or more sulfur atoms (that is, Sₚ, wherein p is an integer that is equal to 3 or higher). As used herein, a “polyselane species” refers to a compound having an unbranched or branched chain of selenium atoms terminating in H: HSeₙH, wherein n is as defined herein, provided n is 2 or more. As used herein, a “polyselenide species” refers to a compound having an unbranched or branched chain of selenium atoms of the following formula: ASeₘB, wherein A and B are independently not H, and m is as defined herein, provided m is 2 or more. As used herein a “hydropolyselenide species” refers to a compound having an unbranched or branched chain of selenium atoms of the following formula: RSeₘH, wherein R is not H and m is as defined herein, provided m is 2 or more. As used herein, a “cyclic polyselenium species” comprises a ring of selenium atoms, wherein the ring may comprise 3 or more selenium atoms (that is, Se_{q}, wherein q is an integer that is equal to 3 or higher). As used herein, a “poly(sulfur/selenium) species” refers to a compound comprising an unbranched, branched, or cyclic chain of both sulfur and selenium atoms, in any combination, analogous to the polysulfur species and polyselenium species described above, provided at least one sulfur and at least one selenium is present. Any of these compounds may comprise a ═S and/or a ═Se group, as appropriate. Any of these compounds may be used with any embodiment described herein, including in combinations as well as in compositions and/or methods. Examples of each of these compounds are well-known in the art. See, e.g., Devillanova, 2006; Steudel, Top. Curr. Chem., vol. 230, 2003a; Steudel, Top. Curr. Chem., vol. 231, 2003b, and U.S. Patent Publn. 60/946,065, each of which is incorporated herein by reference in its entirety.

It is to be noted that compounds or classes or subclasses thereof of the invention that may be ascribed to one type of definition found herein may simultaneously be ascribed to another type of definition found herein.

In any embodiment that employs a polychalcogenide composition, it is contemplated that a polychalcogenide compound, polychalcogenide salt, or compound comprising a Ch-(Ch)ₙ-Ch group may be used, and vice versa, unless specifically noted otherwise. Combinations of these agents are also contemplated. Pharmaceutical compositions may comprise any one or more of these agents as well.

Certain embodiments contemplate methods of making a polychalcogenide composition or polychalcogenide compound, the method comprising: (a) mixing an aqueous solution comprising a peptide or a protein with (b) a polychalcogenide salt. In certain embodiments, the peptide or the protein is stable in the aqueous solution of (a) and may further remain stable once combined with the polychalcogenide salt. The polychalcogenide salt may be a liquid solution, or may be comprised in a liquid solution.

In certain embodiments, the method may be further defined as (a) mixing an aqueous solution comprising the peptide or the protein with (b) a liquid solution formed from mixing two parts sodium sulfide nonahydrate with one part solid S₈, such that an aqueous solution comprising a polysulfide composition or polysulfide compound is prepared. The method may further comprise diluting the liquid solution formed from mixing two parts sodium sulfide nonahydrate with one part solid S₈ with HCl. The concentration of HCl may range from about 10-20 mM, for example. In certain embodiments, the aqueous solution of (a) is adjusted to about pH 7.0 prior to mixing with the liquid solution formed from mixing two parts sodium sulfide nonahydrate with one part solid S₈. The method may be further defined as a method of (a) mixing an aqueous solution comprising a mixture of human serum albumin and a glutathione with (b) a liquid solution formed by mixing two parts sodium sulfide nonahydrate with one part solid S₈. In certain embodiments of this method, the ratio of human serum albumin to total glutathione in (b) ranges from about 2:1 to about 3:1. Any method of making a polychalcogenide compound or polychalcogenide composition may be combined with any other method or embodiment described herein.

The present inventor, though not bound by the following theory, believes that the oxidation state of a chalcogen in an administered polychalcogenide composition, and/or in a metabolized polychalcogenide composition may be responsible for some of the biological effects seen with these compositions. Accordingly, certain aspects of the present invention contemplate polychalcogenide compositions, polychalcogenide compounds, polychalcogenide salts, or compound comprising a Ch-(Ch)ₙ-Ch group, wherein one or more chalcogens is of a certain oxidation state, either before or after they are administered to biological matter. Oxidation states available for the various chalcogens are known, and have also been studied in chalcogenides, such as sulfides. (Steudel, 2003a, Steudel, 2003b, Devillanova, 2006). Sulfur, for example, may exhibit an oxidation state ranging from −2 to +6. In certain embodiments, the oxidation state of at least one chalcogen in a polychalcogenide composition ranges from about, at least about, or at most about −2, −1.9, −1.8, −1.7, −1.6, −1.5, −1.4, −1.3, −1.2, −1, −0.9, −0.8, −0.7, −0.6, −0.5, −0.4, −0.3, −0.2, −0.1, 0, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9 or 6, or any range derivable therein. In any polychalcogenide composition, a terminal chalcogen may independently have an oxidation state of any of these values or ranges. The term “terminal chalcogen” refers to the chalcogen at either end (terminus) of an unbranched or branched chain of chalcogens in a polychalcogenide composition, polychalcogenide compound, polychalcogenide salt, or compound comprising a Ch-(Ch)ₙ-Ch group. In any embodiment herein, each chalcogen may have the same or different oxidation state. Methods of measuring and/or computing the oxidation state of a given atom are well-known to those of skill in the art, both experimentally and computationally. See, e.g., Vairavamurthy, 1998.

In particular embodiments, there is a mixture of polychalcogenide compositions. The mixture may refer to polychalcogenide compositions that have different chalcogens, referred to herein as a “heterogeneous polychalcogenide composition mixture” (e.g., a polysulfide composition and a polyselenide composition) or that contain the same chalcogen element (referred to as a “homogeneous polychalcogenide composition mixture”).

In any embodiment of the present invention, a single polychalcogenide composition may be employed or a mixture of two or more polychalcogenide compositions. In any embodiment wherein a single polychalcogenide composition is employed, two or more polychalcogenide compositions may be employed, and vice versa, unless noted otherwise. Moreover, polychalcogenide compositions may include polychalcogenide salts, in certain embodiments, including salts that are pharmaceutically acceptable. In certain embodiments, a polychalcogenide salt comprises a chalcogen in a −2 oxidation state.

Any polychalcogenide compound, polychalcogenide composition, polychalcogenide salt, or compound comprising a Ch-(Ch)ₙ-Ch group may be excluded from any embodiment described herein; similarly, any combination thereof may be excluded from any embodiment herein.

In certain embodiments, a mixture of different polychalcogenide compounds or salts is purified so that there is no longer a variegated mixture but a purified composition containing purified polychalcogenide compounds or salts or a substantially purified subset of compounds or salts (i.e., >50% enriched with respect to the initial mixture).

Moreover, it will be understood that any polychalcogenide composition, polychalcogenide compound, polychalcogenide salt, or compound comprising a Ch-(Ch)ₙ-Ch group discussed herein can be provided in prodrug form to biological matter, meaning that the biological matter or other substance(s) in the environment of the biological matter alters the prodrug into its active form, that is, into an active polychalcogenide composition, polychalcogenide compound, or polychalcogenide salt. It is contemplated that the term “precursor” covers compounds that are considered “prodrugs.”

The term “biological matter” refers to any living biological material (e.g., mammalian biological material) including cells, tissues, organs, and/or organisms, or any combination thereof. Organisms include a subject, which refers to a higher order mammal, such as a human. Embodiments of the invention may be applied to biological matter, including subjects. Biological matter is described in more detail below, and in any embodiment discussed herein, the biological matter (e.g., cell, tissue, or organ) may be in vitro or in vivo. The term “in vivo biological matter” refers to biological matter that is in vivo, i.e., still within or attached to an organism. Moreover, the term “biological matter” will be understood as synonymous with the term “biological material.”

In some embodiments, tissue is all or part of an organ. The terms “tissue” and “organ” are used according to their ordinary and plain meanings. Though tissue is composed of cells, it will be understood that the term “tissue” refers to an aggregate of similar cells forming a definite kind of structural material. Moreover, an organ is a particular type of tissue. Accordingly, in some embodiments, tissue is all or part of an organ. In some embodiments, a polychalcogenide composition is administered to a subject or is administered more directly to tissue or one or more organs in a subject.

The present invention also concerns methods of providing a polychalcogenide compound, polychalcogenide composition, polychalcogenide salt, and/or compound comprising a Ch-(Ch)ₙ-Ch group to a subject. Such methods may comprise providing a polychalcogenide composition comprising a compound of formula (I), or a compound of formula (IV), as those compounds are described above. The subject may have or is at risk for a hypoxic/ischemic injury, hemorrhagic shock, hyperproliferative disease or condition, neurodegenerative disease, inflammatory disease, transplant rejection, or autoimmune disease or condition, or any other condition or disease described herein.

In particular embodiments, polychalcogenide compositions of the present invention are used to protect biological matter, e.g., cells, tissues, organs, and/or organisms (e.g., a mammal), (i) before or after an injury (e.g., a traumatic injury or surgery) or (ii) before or after the onset or progression of a disease or adverse medical condition. Such methods may comprise providing to the biological matter an effective amount of a polychalcogenide composition prior to, during, and/or following the injury or the onset or progression of the disease or adverse medical condition. In certain embodiments, protective methods may be considered as “pre-treatment” with a polychalcogenide composition. Pre-treatment includes methods wherein biological matter is provided with a polychalcogenide composition (i) before; (ii) both before and during, or (iii) before, during and after biological matter is subjected to adverse conditions (e.g., an injury or onset or the progression of a disease). In certain embodiments, there are methods of protecting a mammal from suffering cellular damage from surgery, a disease or adverse medical condition, comprising providing to the mammal an effective amount of a polychalcogenide composition prior to the surgery, or onset or progression of the disease or condition, that protects the mammal from cellular damage (compared to the damage in the absence of the compound). This embodiment may be used in the context of a variety of different surgeries, diseases and adverse medical conditions, including, e.g., cardiopulmonary surgery, unstable angina, post-angioplasty, aneurism, hemorrhagic stroke or shock, trauma, or blood loss. As used in this or any other method, “surgery” may be elective, planned, or emergency surgery.

In certain embodiments, a polychalcogenide composition is not provided during or after trauma, injury, or the onset or progression of the disease.

Biological matter in need of protection from injury or damage is biological matter in which all or part of the biological matter may yield direct or indirect physiological benefits from being protected. For example, a patient at risk for hemorrhagic shock may be considered in need of a polychalcogenide composition of the invention, or a patient who will undergo coronary artery bypass surgery may benefit from protecting the heart from ischemia/reperfusion injury. Other applications are discussed throughout this disclosure.

In certain embodiments, protective methods may be used to protect cells. Protected cells can be used in a number of applications including, e.g., for transfusion or transplantation (therapeutic applications); for research purposes; for screening assays to identify, characterize, or manufacture compounds that, for example, induce stasis; for testing a sample from which the cells were obtained (diagnostic applications); for preserving or preventing damage to the cells that will be placed back into the organism from which they were derived (preventative applications); or for preserving or preventing damage to cells during transport or storage. Methods of protecting tissues and/or organs may be used for similar purposes, in certain embodiments.

The invention also concerns methods of preventing an organism (e.g., a mammal) from bleeding to death or suffering irreversible tissue damage as a result of bleeding by providing to the organism an amount of a polychalcogenide composition sufficient to prevent the animal from bleeding to death. The terms “bleeding” and “hemorrhaging” are used interchangeably to refer to any discharge of blood from a blood vessel. They include, but are not limited to, internal and external bleeding and bleeding from an injury (which may be from an internal source, or from an external physical source such as from a gunshot, stabbing, physical trauma, etc.). In certain embodiments, there are methods for treating or preventing hemorrhagic shock in a patient comprising providing to the patient an effective amount of a polychalcogenide composition. In particular embodiments, the polychalcogenide composition does not affect blood pressure by more than 10 mm Hg. Such methods may prevent lethality, for example.

Moreover, additional embodiments of the invention concern prevention of death or irreversible tissue damage from blood loss or other lack of oxygenation to cells or tissue, such as from lack of an adequate blood supply. This may be the result of, for example, actual blood loss, or it may be from conditions or diseases that prevent cells or tissue from being perfused (e.g., reperfusion injury), that cause blockage of blood to cells or tissue, that reduce blood pressure locally or overall in an organism, that reduce the amount of oxygen is carried in the blood, or that reduces the number of oxygen carrying cells in the blood. Conditions and diseases that may be involved include, but are not limited to, blood clots and embolisms, cysts, growths, tumors, anemia (including sickle cell anemia), hemophilia, other blood clotting diseases (e.g., von Willebrand, ITP), and atherosclerosis. Such conditions and diseases also include those that create essentially hypoxic or anoxic conditions for cells or tissue in an organism because of an injury, disease, or condition.

It is contemplated that polychalcogenide compositions of the invention may be exposed to an entire organism or a part of an organism (such as in cells, in tissue, and/or in one or more organs), whether that part remains within the organism or is removed from the organism, or the whole organism will be induced into or subjected to a different state or environment (e.g., stasis, or in an environment having less oxygen compared to ambient conditions). In certain embodiments, one or more cells, tissues, or organs is separate from an organism. The term “isolated” can be used to describe such biological matter. Any method described herein employing biological matter may be employed with respect to isolated biological matter, unless specifically noted otherwise. For example, the present invention concerns methods for enhancing survivability of isolated tissue comprising: a) identifying the tissue in which survivability is desired; and b) exposing the tissue to an effective amount of a polychalcogenide composition. In certain embodiments, stasis is induced in isolated biological matter.

In certain other embodiments, polychalcogenide compositions affect metabolism, such as by reducing it compared to normal conditions. It is contemplated that the compounds may act as metabolic alternatives in that they result in the production of exhaled chalcogenide, such as H₂S, instead of H₂O. This is different from a metabolic depressor, such as an oxygen antagonist or stasis inducer, as discussed in U.S. Patent Publns. 2005/0053912, 2005/0170019, 2005/0147692, 2005/0136125, 2008/0085329, and 2007/0078113, and U.S. patent application Ser. No. 11/738,294, all of which are hereby incorporated by reference in their entireties. Nonetheless, any of the active compounds discussed in these patent applications (oxygen antagonists or otherwise) may be used in conjunction with polychalcogenide compositions in any embodiment of the invention.

Without being bound by theory, it is contemplated that in some embodiments of the invention, a polychalcogenide composition acts as a blood substitute. Accordingly, the present invention contemplates methods for treating a subject with a blood substitute comprising administering to the subject an effective amount of a polychalcogenide composition. Also contemplated are methods of contacting a cell, tissue, or organ with a blood substitute, comprising administering to the cell, tissue, or organ an effective amount of a polychalcogenide composition.

The terms “contacted” and “exposed,” when applied to biological matter (e.g., cells) are used herein to describe the process by which a polychalcogenide composition, polychalcogenide compound, polychalcogenide salt, or compound comprising a Ch-(Ch)ₙ-Ch group is delivered to target biological matter or is placed in direct juxtaposition with the target biological matter. More particularly, the term “expose” is used according to its ordinary meaning to indicate that biological matter as a whole is subjected to a polychalcogenide composition, polychalcogenide compound, polychalcogenide salt, or compound comprising a Ch-(Ch)ₙ-Ch group. This can be achieved in some embodiments by contacting biological matter as a whole with a polychalcogenide composition. In the case of in vivo cells, tissues, or organs, “expose” may further mean “to lay open” the material so that it can be contacted with a polychalcogenide composition. This can be done, for example, surgically or via incubation, immersion, perfusion, infusion, injection, or topical application. In addition, if it desirable to implement methods with respect to an entire organism, inhalation or ingestion of the polychalcogenide composition, or any other route of administration discussed herein is contemplated for use with polychalcogenide compositions. Furthermore, the term “provide” is used according to its ordinary and plain meaning to mean “to supply.” The term “provide” encompasses the term “expose” in the context of the term “effective amount,” according to the present invention. In the case of patients, the term “provide” may refer to the action performed by a doctor or other medical personnel who prescribes a particular polychalcogenide composition, polychalcogenide compound, polychalcogenide salt, or compound comprising a Ch-(Ch)ₙ-Ch group, or administers it directly to the patient.

The present invention also covers a method comprising administering to biological matter (e.g., a mammal) an effective amount of a polychalcogenide composition to achieve a particular result. The term “effective amount” means an amount that can achieve the stated result. In certain methods of the invention, an “effective amount” is an amount that protects biological matter from injury or damage. In other methods, an “effective amount” is an amount that staves off tachycardia in a subject at risk for death from hemorrhagic shock. In additional embodiments, an “effective amount” may refer to an amount that increases the survivability of biological matter. This can be determined (or assumed) based on comparison or previous comparison to untreated biological matter or biological matter treated with a different dosage or regimen that does not experience a difference in survivability. In some instances, an effective amount is an effective amount to alter metabolism. In specific embodiments, an effective amount induces stasis.

In some embodiments of the invention, an effective amount may be expressed as LD₅₀, which refers to the “median lethal dose,” which means the dose that is administered that kills half the population of animals (causes 50% mortality). In other embodiments, an effective amount exceeds what is considered a lethal concentration.

In some embodiments, an effective amount is characterized as a sublethal dose of a polychalcogenide composition. In the context of enhancing survivability of or preventing injury or damage to cells, tissues, or organs (not the whole organism), a “sublethal dose” means a single administration of the polychalcogenide composition that is less than half of the amount of the polychalcogenide composition that would cause at least a majority of cells in a biological matter to die within 24 hours of the administration. If enhancing survivability of or preventing injury or damage of the entire organism is desired, then a “sublethal dose” means a single administration of the polychalcogenide composition that is less than half of the amount of the polychalcogenide composition that would cause the organism to die within 24 hours of the administration. In other embodiments, an effective amount is characterized as a near-lethal dose of the polychalcogenide composition. Similarly, in the context of enhancing survivability of or preventing injury or damage to cells, tissues, or organs (not the whole organism), a “near lethal dose” means a single administration of the polychalcogenide composition that is within 25% of the amount of the inhibitor that would cause at least a majority of cell(s) to die within 24 hours of the administration. If enhancing survivability of or preventing injury or damage of the entire organism is desired, then a “near lethal dose” means a single administration of the polychalcogenide composition that is within 25% of the amount of the inhibitor that would cause the organism to die within 24 hours of the administration. In some embodiments a sublethal dose is administered by administering a predetermined amount of the polychalcogenide composition to the biological material.

In certain embodiments, biological matter is exposed to or provided with a polychalcogenide composition in an amount that exceeds what was previously understood to be the maximum tolerated dose before the onset of adverse physiological ramifications, such as apnea, lack of observable skeletal muscle movement, dystonia, and/or hyperactivity. Such an amount may be relevant to increasing survivability in some embodiments of the invention, for instance, to increase the chances of surviving adverse conditions, such as those that would induce death from hemorrhagic shock.

Biological matter may, for example, be exposed to or contacted with more than one polychalcogenide composition. For example, biological matter may be exposed to at least one polychalcogenide composition, including 2, 3, 4, 5, 6, 7, 8, 9, 10 or more polychalcogenide compositions, or any range derivable therein. With multiple compositions, the term “effective amount” refers to the collective amount of the compositions. In certain embodiments, the biological matter may be exposed to a first polychalcogenide composition and then exposed to a second polychalcogenide composition simultaneously, in an overlapping fashion, or separately.

For any method described herein, single or multiple dosages of a polychalcogenide composition are contemplated. The amount of the polychalcogenide composition that is provided to biological matter can be about, at least about, or at most about 0.001, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, 9900, 10000, 11000, 12000, 13000, 14000, 15000, 16000, 17000, 18000, 19000, 20000, 21000, 22000, 23000, 24000, 25000, 26000, 27000, 28000, 29000, 30000, 31000, 32000, 33000, 34000, 35000, 36000, 37000, 38000, 39000, 40000, 41000, 42000, 43000, 44000, 45000, 46000, 47000, 48000, 49000, 50000, 60000, 70000, 80000, 90000, 100000, 110000, 120000, 130000, 140000, 150000, 160000, 170000, 180000, 190000, 200000, 210000, 220000, 230000, 240000, 250000, 260000, 270000, 280000, 290000, 300000, 310000, 320000, 330000, 340000, 350000, 360000, 370000, 380000, 390000, 400000 mg, mg/kg (biological matter weight), mmol/kg (biological matter weight), mg/m², mM, M, ppm, or ppb, or any range derivable therein. Molar concentration may be readily converted into weight per volume, as known in the art. An effective amount of a polychalcogenide composition may involve any of these values, or any range of these values.

Moreover, a dosage or effective amount can be expressed as a concentration with or without a qualification on length of time of exposure. In any method described herein, biological matter may be exposed to a polychalcogenide composition for about, at least about, or at most about 5, 10, 15, 20, 25, 30, 35, 40, 45, 50, 55, 60 seconds, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 minutes, 1, 2, 3, 4, 5, 6, 7, 8, 9, 10, 11, 12, 13, 14, 15, 16, 17, 18, 19, 20, 21, 22, 23, 24 hours, 1, 2, 3, 4, 5, 6, 7 days, 1, 2, 3, 4, 5 weeks, 1, 2, 3, 4, 5, 6, 7, 8, 9, 10, 11, 12 months, 1, 2, 3, 4, 5, 6, 7, 8, 9, 10, 11, 12, 13, 14, 15, 16, 17, 18, 19, 20 or more years, and any combination or range derivable therein. These time periods may be employed with respect to the administration of any amount of polychalcogenide composition described herein. It is further contemplated that the amount of exposure time may be indefinite, depending on the reason or purpose for administering the polychalcogenide composition. Thereafter, biological matter may continue to be exposed to the polychalcogenide composition, or, in other embodiments of the invention, the biological matter may no longer be exposed to the polychalcogenide composition. This latter step can be achieved either by removing the polychalcogenide composition from the presence of the biological matter, or the biological matter may be removed from an environment containing the polychalcogenide composition. Additionally, matter may be exposed to or provided with any polychalcogenide composition continuously (a period of time without a break in exposure), intermittently (exposure on multiple occasions), or on a periodic basis (exposure on multiple occasions on a regular basis). It is contemplated that biological matter may be exposed to one or more polychalcogenide compositions one or more times. For example, biological matter may be exposed to one or more polychalcogenide compositions 1, 2, 3, 4, 5, 6, 7, 8, 9, 10 or more times, meaning when a biological matter is exposed multiple times that there are periods of respite (with respect to exposure to the polychalcogenide composition) in between. In certain embodiments, such exposure may begin before, during, and/or after initial damage, such as trauma, a wound or degeneration, occurs.

Furthermore, in some embodiments of the invention, biological matter is exposed to or provided with a polychalcogenide composition for a sustained period of time, where “sustained” means a period of time of at least about 2 hours. Moreover, in certain embodiments, a polychalcogenide composition may be provided on a continuously sustained basis at level that is considered “low,” meaning a level that is less than the amount that causes metabolic flexibility such as seen with drop in CBT, heart rate, or CO₂ or O₂ consumption or production.

In certain embodiments, a subject is provided with a polychalcogenide composition in an amount and for a time that protects the subject from damage or death resulting from an injury or the onset or progression of a disease. This or any other method described herein may comprise or further comprise exposing biological matter (e.g., a subject) to a controlled temperature and/or pressure environment.

In some embodiments, biological matter is exposed to or provided with a polychalcogenide composition at least before and during; before, during, and after; during and after; or solely after a particular injury, trauma (for instance, surgery), treatment, adverse condition, or other relevant event or situation, such as those described herein. For example, the polychalcogenide composition may be provided to a subject after blood loss and until fluids can be replenished and/or the risk of hemorrhagic shock subsides.

In certain embodiments, the subject becomes apneic upon exposure to a polychalcogenide composition, which is marked by a cessation in breathing and then an apnic breath after a short period of time. “Apnea” refers to the period of time during which breathing is markedly reduced such that the subject takes 10% or fewer number of breaths. Thus, it is contemplated that a subject induced into apnea may exhibit 0, 1, 2, 3, 4, 5, 6, 7, 8, 9, 10% the number of breaths subsequent to exposure to a polychalcogenide composition. In certain embodiments of the invention, apnea continues until the subject is no longer exposed to the polychalcogenide composition. In certain embodiments regarding apnea or any other condition described herein, the organism also does not exhibit any skeletal muscle movement as a result of exposure to or administration of a polychalcogenide composition.

In some cases, biological matter is identified or determined to be in need of a polychalcogenide composition based on one or more tests, screens, or evaluations that indicate a condition or disease, or the risk of a condition or disease, that can be prevented or treated by a polychalcogenide composition. For example, the taking of a patient medical or family medical history (patient interview) may yield information that biological matter is in need of protection because it is at risk of injury or damage. Moreover, a patient may be determined to be in need of enhanced survivability or treatment based on methods discussed herein, such as by taking a patient or family medical history. As such, in certain embodiments, there is a step of identifying a subject in need of treatment. In particular embodiments, this may involve determining that a subject is at risk for hemorrhagic shock or other ischemic/hypoxic injury. In other embodiments, there is a step of observing apnea in the organism. In even further embodiments, such methods involve obtaining a blood sample from the organism and/or evaluating the organism's blood, such as by analyzing its color. Methods of such blood analyses are known in the art. Accordingly, optional steps for any of the methods of the invention include identifying an appropriate polychalcogenide composition; diagnosing a patient; taking a patient history, and/or having one or more tests done on the patient prior to administering or prescribing a polychalcogenide composition to the patient.

In further embodiments, after exposure to a polychalcogenide composition the amount of compound that is metabolized by biological matter can be measured or monitored. In certain embodiments, the amount of chalcogenide that is exhaled or released by a subject is measured or monitored to evaluate dosage or metabolic rate of the polychalcogenide composition. Measurements can be taken once or multiple times on a periodic or sustained basis. Other physiological parameters of biological matter may be measured, monitored, or assessed. Such measurements or assessments can be done before, during, and/or after each administration of a polychalcogenide composition or after a treatment regimen with a polychalcogenide composition. It is contemplated that one or more of the following may be assessed, monitored, and/or measured: heart rate (pulse), mean arterial pressure, blood pressure, brain activity, respiration rate, metabolism, toxicity, consciousness, and/or other biotelemetric parameters. Monitoring methods include, but are not limited to, monitoring the amount and/or administration duration of a polychalcogenide composition, monitoring a physiological response (e.g., pulse, respiration, pain response, movement or motility, metabolic parameters such as cellular energy production or redox state, etc.) of the biological matter to the administration of the polychalcogenide composition, and/or reducing, interrupting or ceasing administration of the compound(s) when a predetermined floor or ceiling for a change in that response is measured, etc. Methods of performing these and other measuring and monitoring steps are well-known in the art. Each of these methods may be combined. Moreover, any one or more of these steps can be employed in any method of the invention.

Compositions, methods, and articles of manufacture of the invention can be used on biological matter that will be transferred back into a donor organism from which the matter was derived (autologous) or a different recipient (heterologous) subject. In some embodiments, biological matter is obtained directly from a donor organism. In others, the biological matter is placed in culture prior to exposure to a polychalcogenide composition. Moreover, methods include administering or implanting the biological matter that was exposed to the polychalcogenide composition to a live recipient organism. In some embodiments, an organ or tissue to be retrieved and then transplanted is exposed to the polychalcogenide composition while still in the donor subject.

Methods of the invention also concern enhancing survivability or preventing damage or injury of a cell comprising contacting the cell with a polychalcogenide composition that creates hypoxic conditions for an effective amount of time for the cell to be protected. Methods of the invention also concern enhancing survivability or preventing damage or injury of isolated tissue or an organ comprising incubating the tissue or organ with a polychalcogenide composition that creates hypoxic conditions for an effective amount of time for the tissue or organ to be protected.

In certain embodiments, biological matter (e.g., a subject) is provided with a polychalcogenide composition under hypoxic or anoxic conditions or prior to exposure to hypoxic or anoxic conditions and in particular embodiments, the hypoxic or anoxic conditions would damage the biological matter in the absence of the compound. In other embodiments, the biological matter is not exposed to the compound during exposure to hypoxic or anoxic conditions. Such conditions are described herein.

The present invention also provides methods, compositions, and apparatuses for enhancing survivability of and/or reducing damage to biological matter under adverse conditions by reducing metabolic demand, oxygen consumption or requirements, temperature, or any combination thereof. A polychalcogenide composition may, for example, enhance survivability by preventing or reducing damage to the biological matter, preventing all or part of the matter from dying or senescing, and/or extending the lifespan of all or part of the biological matter, relative to biological matter not exposed to the compound. Alternatively, in some embodiments the polychalcogenide composition prolongs survival of tissue and/or an organism that would otherwise not survive without the compound. In certain embodiments methods involve an injury or disease that is associated with a reduction in metabolism or temperature of the subject.

It is contemplated that embodiments of the invention may be discussed in terms of a reduction in the rate or amount of oxygen consumption by the biological matter at about, at least about, or at most about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% or more, or any range derivable therein. This can also be expressed and assessed in terms of any cellular respiration indicator. Administration of a polychalcogenide composition may reduce the rate or amount of oxygen consumption by biological matter. The present invention also covers reducing the oxygen requirement of biological matter, meaning that the amount of oxygen required by the biological matter to survive is reduced. It is generally known how much oxygen particular biological matter require to survive, which can also be dependent on time, pressure, and/or temperature. In certain embodiments of the invention, the oxygen requirement of the biological matter is reduced by about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100%, or any range derivable therein, as compared to the requirement of the biological matter in the absence of the effective amount of the polychalcogenide composition(s). Methods of measuring oxygen consumption and requirements are well-known in the art.

Without being bound by theory, the inventor postulates that the administration of a polychalcogenide composition, such as a polysulfide composition, results in the production of H₂S which, in turn, may result in the depression of metabolism, such as a reduction in cellular respiration. Accordingly, in some embodiments of the invention, methods are provided for reducing cellular respiration, which may or may not be as high as that needed to reach stasis.

Survivability includes survivability when the matter is under adverse conditions—that is, conditions under which there can be adverse and nonreversible damage or injury to biological matter. Adverse conditions can include, but are not limited to, when oxygen concentrations are reduced in the environment (hypoxia or anoxia, such as at high altitudes or under water); when the biological matter is incapable of receiving that oxygen (such as during ischemia), which can be caused by i) reduced blood flow to organs (e.g., heart, brain, and/or kidneys) as a result of blood vessel occlusion (e.g., myocardial infarction, and/or stroke), ii) extracorporeal blood shunting as occurs during heart/lung bypass surgery (e.g., “pumphead syndrome” in which heart or brain tissue is damaged as a result of cardiopulmonary bypass), or iii) as a result of blood loss due to trauma (e.g., hemorrhagic shock or surgery); hypothermia, where the biological material is subjected to sub-physiological temperatures, due to exposure to cold environment or a state of low temperature of the biological material, such that it is unable to maintain adequate oxygenation of the biological materials; hyperthermia, whereby temperatures where the biological material is subjected to supra-physiological temperatures, due to exposure to hot environment or a state of high temperature of the biological material such as by a malignant fever; conditions of excess heavy metals, such as iron disorders (genetic as well as environmental) such as hemochromatosis, acquired iron overload, sickle-cell anemia, juvenile hemochromatosis African siderosis, thalassemia, porphyria cutanea tarda, sideroblastic anemia, iron-deficiency anemia and anemia of chronic disease. In other embodiments of the invention, one or more compounds may be used to increase or enhance survivability of biological matter; reversibly inhibit the metabolism and/or activity of biological matter; reduce the oxygen requirement of biological matter; reduce or prevent damage to biological matter under adverse conditions; prevent, treat, and/or reduce damage or injury to biological matter; prevent aging or senescence of biological matter; and/or, provide a therapeutic benefit as described throughout the application with respect to polychalcogenide compositions. Damage may occur from trauma and/or an adverse condition, as described herein.

Polychalcogenide compositions may lead to or provide desired effect(s), in some embodiments, only when they are in the context of the biological matter (i.e., have no lasting effect) and/or they may provide for these effect(s) for more than 24 hours after the biological matter is no longer exposed to it. Moreover, this can also be the case when a combination of polychalcogenide compositions is used.

In certain embodiments, methods including pre-exposure to a polychalcogenide composition (i.e., pre-treatment) are used to treat conditions in which an injurious or disease insult is 1) scheduled or elected in advance, or 2) predicted in advance to likely occur. Examples meeting condition 1 include, but are not limited to, surgery where blood loss may occur spontaneously or as a result of a procedure, cardiopulmonary bypass in which oxygenation of the blood may be compromised or in which vascular delivery of blood may be reduced (as in the setting of coronary artery bypass graft (CABG) surgery), or in the treatment of organ donors prior to removal of donor organs for transport and transplantation into a recipient in need of an organ transplant. Examples meeting condition 2 include, but are not limited to, medical conditions in which a risk of injury or disease progression is inherent (e.g., in the context of unstable angina, following angioplasty, bleeding aneurysms, hemorrhagic strokes, following major trauma or blood loss), or in which the risk can be diagnosed using a medical diagnostic test.

Biological matter may be provided with or exposed to a polychalcogenide composition through inhalation, injection, catheterization, immersion, perfusion, topical application, absorption, or adsorption. Moreover, biological matter may be provided with or exposed to an active compound by administration to the biological matter intravenously, intradermally, intraarterially, intraperitoneally, intralesionally, intracranially, intraarticularly, intraprostaticaly, intrapleurally, intratracheally, intranasally, intrathecally, intravitreally, intravaginally, intrarectally, topically, intratumorally, intramuscularly, intraperitoneally, intraocularly, subcutaneously, subconjunctival, intravesicularlly, mucosally, intrapericardially, intraumbilically, intraocularally, orally, topically, locally, by inhalation, by injection, by infusion, by continuous infusion, by localized perfusion, via a catheter, or via a lavage. It is specifically contemplated that in some embodiments a polychalcogenide composition is provided to a subject by nebulizer, injection, or infusion. Such methods may be applied with any embodiment of the invention. For example, 0.1 mL per hour can be injected; moreover, there can be injections every minute, or with repeated boluses requested by the patient up to maximum number per hour, or a variable number of injections.

It is specifically contemplated that any subset of polychalcogenide compositions identified by name or structure herein may be used in methods, compositions and articles of manufacture. It is also specifically contemplated that any subset of these compounds may be disclaimed as not constituting embodiments of the invention. The present invention also concerns pharmaceutical compositions comprising an effective amount of one or more polychalcogenide compositions. It is understood that such pharmaceutical compositions are formulated as pharmaceutically acceptable compositions. For example, the composition may include a pharmaceutically acceptable carrier. In any embodiment herein that employs a polychalcogenide composition, a pharmaceutical composition comprising a polychalcogenide composition may be employed, unless noted otherwise.

The polychalcogenide composition may be provided as a gas, semi-solid liquid (such as a gel or paste), liquid, or solid. Biological matter may be exposed to a polychalcogenide composition in more than one state. Moreover, the polychalcogenide composition may be formulated for a particular mode of administration using techniques known in the art. In certain embodiments, a composition comprises a gaseous, semi-solid liquid, liquid, or solid form of one or more polychalcogenide compositions.

In certain embodiments, the effective amount of a gas(es) may be expressed as about, at least about, or at most about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%, or any range derivable therein, with respect to the concentration in the air to which the biological matter is exposed. These amounts may be used in any method described herein.

In certain embodiments, a composition has one or more polychalcogenide compositions as a gas that is bubbled in it so that the composition provides the compound to the biological matter when it is exposed to the composition. Such compositions may be pharmaceutical compositions. Such compositions may be gels, liquids, or other semi-solid material. In certain embodiments, a solution has a polychalcogenide composition as a gas bubbled through it. It is contemplated that the amount bubbled in the gas will provide the appropriate amount of the compound to biological material exposed to the solution.

In some cases, the pharmaceutical composition is a medical gas. According to the United States Food and Drug Administration, “medical gases” are those gases that are drugs within the meaning of §201(g)(1) of the Federal Food, Drug and Cosmetic Act (“the Act”) (21 U.S.C. §321(g) and pursuant to §503(b)(1)(A) of the Act (21 U.S.C. §353(b)(1)(A) are required to be dispensed by prescription. As such, such medical gases require an appropriate FDA label. In the context of the present invention, a medical gas includes at least one polychalcogenide composition.

Biological matter is exposed to the gas in a closed container in some embodiments of the invention. In some cases, the closed container can maintain a particular environment or modulate the environment as is desired. The environment refers to the amount of polychalcogenide composition that the biological matter is exposed and/or the temperature, gas composition, or pressure of the environment.

In certain embodiments, the environment containing the biological matter cycles at least once to a different amount or concentration of the polychalcogenide composition, wherein the difference in amount or concentration is by at least one percent. The environment may cycle back and forth between one or more amounts or concentrations of the polychalcogenide composition, or it may gradually increase or decrease the amount or concentrations of that compound. In some cases, the different amount or concentration is between about 0 and 99.9% of the amount or concentration of the polychalcogenide composition to which the biological matter was initially exposed. It is contemplated that the difference in amount and/or concentration is about, at least about, or at most about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% or more, or any range derivable therein.

The present invention further comprises apparatuses and articles of manufacture comprising packaging material and, contained within the packaging material, a polychalcogenide composition, wherein the packaging material comprises a label that indicates that it can be used for protecting biological matter or enhancing survivability or any other method of the invention, and optionally directions for use or administration. The present invention also concerns an article of manufacture comprising packed together: a polychalcogenide composition and instructions for use, comprising: (a) identifying biological matter in need of protection or treatment; and (b) administering an effective amount of the polychalcogenide composition to the biological matter.

The present invention also concerns kits and methods of using these kits. Kits may comprise any article of manufacture described herein, for example.

In certain other embodiments, any toxic effect on the biological matter from administration of a polychalcogenide composition is monitored or controlled for. It is contemplated that a skilled artisan is aware of a number of ways of evaluating toxicity effects in biological matter.

Other aspects of the invention concern methods for preserving in vivo biological matter comprising exposing the in vivo biological matter to an effective amount of a polychalcogenide composition to preserve the biological matter in vivo.

In another embodiment, there is a method of anesthetizing an organism comprising exposing biological matter in which anesthesia is desired to an effective amount of a polychalcogenide composition. It is contemplated that the anesthesia may be similar to local or general anesthesia.

The present invention further includes methods of protecting a mammal from radiation therapy or chemotherapy comprising contacting the mammal with an effective amount of a polychalcogenide composition prior to or during radiation therapy or chemotherapy. A localized part of the mammal may be contacted with the polychalcogenide composition, for example.

In certain embodiments, there are methods of treating a hyperproliferative disease (e.g., cancer) in a mammal comprising contacting the mammal with an effective amount of a polychalcogenide composition and subjecting the mammal to hyperthermia therapy.

In some cases, the invention concerns a method for (i) inducing cardioplegia in a patient undergoing bypass surgery, (ii) preventing hematologic shock in a patient, (iii) promoting wound healing in an organism, (iv) preventing or treating neurodegeneration in a mammal, (v) inducing hibernation in a mammal, or (vi) treating cyanide poisoning in a subject, comprising administering to the patient, organism, mammal, or subject an effective amount of a polychalcogenide composition. Regarding (i) in particular, it is contemplated that administration may be local to the heart so as to protect it. Such local administration may be employed in other methods as well regarding the heart or any other organ (or cell or tissue).

In certain embodiments, there are methods for treating a patient affected with a hematological disorder, which means a disease, disorder or condition that affects any hematopoietic cells or tissue, comprising administering to the patient an effective amount of a polychalcogenide composition. Non-limiting examples of these disorders include sickle cell disease and thalassemia. In other embodiments, there are methods for enhancing survivability in a patient with cystic fibrosis (CF) by administering or providing an effective amount to the patient of a polychalcogenide composition.

The present invention also concerns a method of delaying or reducing trauma and/or the effects of trauma on or in an organism comprising exposing biological matter at risk for trauma to an effective amount of a polychalcogenide composition.

It may be useful to provide additional stimuli to biological matter before withdrawing a polychalcogenide composition. For example, one may subject an animal to increased ambient temperature prior to removing the polychalcogenide composition.

It is of course understood that any method of treatment can be used in the context of a preparation of a medicament for the treatment of or protection against the specified disease or condition. This includes, but is not limited to, the preparation of a medicament for the treatment of ischemic/hypoxic injury, hemorrhagic or hematologic shock, wounds and tissue damage, hyperthermia, hypothermia, neurodegeneration, sepsis, cancer, and/or trauma. Moreover, the invention includes, but is not limited to, the preparation of a medicament for a treatment to prevent death, shock, trauma, organ or tissue rejection, damage from cancer therapy, neurodegeneration, and wound or tissue damage.

As used herein, a “counterion” refers to a positively charged species that counters a negatively charged species. In certain embodiments, a counterion is a positively charged +1 ion. In certain embodiments, the counterion is not H. Non-limiting examples include alkali metal ions, such as Na⁺, K⁺ and Cs⁺. Another non-limiting example of a counterion is ammonium (NH₄⁺).

The invention also encompasses salts of any of the compounds of the present invention, including polychalcogenide compositions. The term “salt(s)” as used herein, is understood as being acidic and/or basic salts formed with inorganic and/or organic acids and bases. Zwitterions (internal or inner salts) are understood as being included within the term “salt(s),” as are quaternary ammonium salts such as alkylammonium salts. Salts include, but are not limited to, sodium, lithium, potassium, amines, tartrates, citrates, hydrohalides, phosphates and the like. Pharmaceutically acceptable salts are encompassed by “salts” as well.

As used herein, the term “alkyl” refers to a straight-chain, branched-chain or cyclic hydrocarbon radical containing from 1 to 10, such as 1 to 6, or 1 to 4, carbon atoms (e.g., 1, 2, 3, 4, 5, 6, 7, 8, 9, or 10 carbon atoms, or any range derivable therein) and may comprise single or double bonds. Examples of such radicals include, but are not limited to, methyl, ethyl, n-propyl, isopropyl, cyclopropyl, n-butyl, isobutyl, sec-butyl, tert-butyl, pentyl, cyclopentyl, iso-amyl, hexyl, cyclohexlyl, decyl, ethenyl, propylenyl, 1,4-butadienyl and the like. In certain embodiments, C1-C6 alkyl groups are contemplated. The term “lower alkyl” is meant to refer alkyl radicals of 1, 2, 3, 4, 5, or 6 carbon atoms, or any range derivable therein.

As used herein, the term “peptide” refers to a compound comprising 2-50 amino acids covalently joined together, typically through an amide bond. In certain embodiments, a peptide is a di-, tri-, tetra- or pentapeptide. The term “amino acid” refers to any of the naturally occurring amino acids, as well as synthetic analogs (e.g., D-stereoisomers of the naturally occurring amino acids, such as D-threonine) and derivatives thereof. α-Amino acids comprise a carbon atom to which is bonded an amino group, a carboxyl group, a hydrogen atom, and a distinctive group referred to as a “side chain.” Amino acids comprising an additional methylene group in their backbone are often called β-amino acids. The side chains of naturally occurring amino acids are well known in the art and include, for example, hydrogen (e.g., as in glycine), alkyl (e.g., as in alanine, valine, leucine, isoleucine, proline), substituted alkyl (e.g., as in threonine, serine, methionine, cysteine, aspartic acid, asparagine, glutamic acid, glutamine, arginine, and lysine), arylalkyl (e.g., as in phenylalanine and tryptophan), substituted arylalkyl (e.g., as in tyrosine), and heteroarylalkyl (e.g., as in histidine). Unnatural amino acids are also known in the art, as set forth in, for example, Williams (1989); Evans et al. (1990); Pu et al. (1991); Williams et al (1991); and all references cited therein. The present invention includes the side chains of unnatural amino acids as well. In certain embodiments, a peptide comprises a chalcogen, such as a sulfur atom. In certain embodiments, a peptide comprises an “exposed” chalcogen, meaning that the chalcogen is available to interact with another atom, such as via a covalent (e.g., disulfide) or ionic bond. Methods of determining whether a peptide comprises an exposed chalcogen are well-known in the art, such as through x-ray crystallography or nuclear magnetic resonance (NMR).

A “protein,” as used herein, comprises at least 50 amino acids covalently joined together, typically through amide bonds, and refers to any protein, including, but not limited to peptides, enzymes, glycoproteins, hormones, receptors, antigens, antibodies, growth factors, etc., without limitation. In certain embodiments, a protein comprises a chalcogen, such as a sulfur atom. In certain embodiments, a protein comprises an “exposed” chalcogen, as that term is described above. Methods of determining whether a protein comprises an exposed chalcogen are well-known in the art, such as through x-ray crystallography or NMR.

The embodiments in the Examples section are understood to be embodiments of the invention that are applicable to all aspects of the invention.

It is contemplated that any embodiment discussed in this specification can be implemented with respect to any method or composition of the invention, and vice versa. Furthermore, compositions of the invention can be used to achieve methods of the invention.

The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the standard deviation of error for the device or method being employed to determine the value. In any embodiment discussed in the context of a numerical value used in conjunction with the term “about,” it is specifically contemplated that the term about can be omitted.

Following long-standing patent law, the words “a” and “an,” when used in conjunction with the word “comprising” in the claims or specification, denotes one or more, unless specifically noted.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 Graph depicting heart rate (HR) and mean arterial pressure (MAP) of a pig undergoing infusion with a polychalcogenide composition.

FIG. 2 Blow-up of graph of FIG. 1 with focus on minutes 20-45 along with H₂S exhalation (vH2S) monitoring.

FIG. 3 Kaplan Meier graph showing survival of pigs treated with a polychalcogenide composition after lethal hemorrhage. Blood was removed from pigs according to the hemorrhage protocol detailed in Example 5. After hemorrhage, pigs were administered 0.325 ml/kg control solution (comprising albumin and glutathione) or test solution (comprising albumin, glutathione and sodium polysulfide oil). See Example 5 for amounts administered. Vertical lines represent death of an animal. Survivors were resuscitated at 180 minutes with shed blood. 83% of treated pigs survive to resuscitation (p=0.048).

FIG. 4 Rats exhale H2S after elemental sulfur administration. Rats were administered elemental colloidal sulfur in the amounts indicated through a femoral vein catheter as a 0.3 ml/kg solution.

FIG. 5 Pigs exhale H2S when administered elemental sulfur. Elemental colloidal sulfur was administered through a venous catheter as a constant infusion in the amounts indicated. H2S values represent the concentration of H2S exhaled after 10 minutes of infusion.

FIG. 6 Pigs exhale H2S when administered sodium polysulfide oil. Sodium polysulfide oil was administered though a venous catheter as a constant infusion in the amounts indicated. The concentration of H2S shown on the y-axis is the value measured after 3 minutes of infusion.

FIG. 7 Table showing experiments regarding survival of pigs after lethal hemorrhage using H2S and various polysulfide compounds and compositions. The data in this table show that various polysulfide formulations can protect pigs from lethal hemorrhage.

FIG. 8 Survival of pigs after lethal hemorrhage and treatment with various polysulfide compositions. Amounts administered are provided in Examples 8 and 9.

FIG. 9 Sodium sulfide is oxidized to a polysulfide by albumin. See Example 10 for details.

### DESCRIPTION OF ILLUSTRATIVE EMBODIMENTS

### I. Polychalcogenide Compositions, Compounds and Salts

As noted above, a “polychalcogenide composition” comprises (i) a peptide or a protein and (ii) a polychalcogenide salt. A “polychalcogenide salt” is a compound of 2 or more consecutive chalcogens, wherein each chalcogen may be the same or different, wherein at least one of the chalcogens is in ionic form, and having at least one counterion. A “polychalcogenide compound” refers to a compound comprising an unbranched, branched, or cyclic chain of 2 or more consecutive chalcogens, wherein each chalcogen may be the same or different, and wherein the compound comprises at least one peptide or protein. A compound comprising a Ch-(Ch)ₙ-Ch group is also described herein, wherein each Ch is an atom that is a chalcogen, each Ch may be the same or different, and each terminal Ch may independently comprise another atom or group or may not, and n is a number or an average number ranging from 1-35, including integers and non-integers, or any other value of n described herein. The oxidation state of any chalcogen in any of these classes of compounds may be of any value described herein.

One may prepare a polychalcogenide composition, polychalcogenide compound, polychalcogenide salt, or compound comprising a Ch-(Ch)ₙ-Ch group by, for example, the methods described below in the Examples section. In certain embodiments, one may combine a polychalcogenide salt with a peptide or protein of interest to generate a polychalcogenide composition or polychalcogenide compound. Methods of preparing polychalcogenide salts are well-known in the art. Steudel and references therein provides examples of preparing such salts having consecutive sulfurs (Steudel, 2003a; Steudel, 2003b). One of the methods provided therein is recited in the Examples section below regarding the preparation of sodium polysulfide oil. This method may be adapted to preparing sodium polyselenide oil as well by, for example, substitution with sodium selenide and elemental selenium. Methods of preparing selenosulfide reagents are also known. (Guo and Zhang, 2000). Arisawa and Yamaguchi (2003) also demonstrate methods of salts having consecutive sulfurs, seleniums and telluriums. Non-limiting examples of polychalcogenide salts that may be combined with a peptide or protein include, for example, Na₂S₂, Na₂S₃, Na₂S₄, Na₂S₆, Na₂Se₂, Na₂Se₆, In₂Te₅, sodium methyl polysulfide, sodium ethyl polysulfide, sodium propyl polysulfide, sodium allyl polysulfide, sodium sulfotrithioite, and potassium ion and cesium ion counterparts of the sodium-containing species. Any of these reagents may be combined as described below in the Examples section with a peptide or protein of interest to generate a polychalcogenide compound or polychalcogenide composition.

In certain embodiments, a polychalcogenide composition comprises a chalcogenide that becomes exposed, as that term is described above, or available once it is taken up by the biological matter. In this respect, the polychalcogenide composition is similar to a prodrug. Therefore, one or more sulfur, selenium, oxygen, tellurium, or polonium atoms in the composition becomes exposed or available subsequent to exposure of the biological matter to the polychalcogenide composition. In this context, “available” means that the sulfur, selenide, oxygen, tellurium, or polonium atom(s) will retain a negative charge.

Polychalcogenide compositions can be toxic, and at some levels lethal, to mammals or other biological matter. In accordance with the present invention, it is anticipated that the levels of a polychalcogenide composition should not exceed lethal levels in the appropriate environment.

### II. Biological Matter

Biological matter contemplated for use with the present invention includes material derived from invertebrates and vertebrates, including mammals. Biological material includes cells, tissues, organs and organisms. In addition to humans, the invention may be employed with respect to mammals of veterinary or agricultural importance including those from the following classes: canine, feline, equine, bovine, ovine, murine, porcine, caprine, rodent, lagomorph, lupine, and/or ursine. The invention also extends to fish and birds. In certain embodiments, animal classes include mammal, reptile, amphibian, bird, fish, invertebrates, and/or fungi. Biological material may be obtained from, for example, plants, protists, and/or prokaryotes. Other examples of sources from which biological matter may be obtained are listed in U.S. Patent Publns. 2005/0170019 and 2007/0078113, each of which is incorporated herein by reference in its entirety.

Biological matter may be a patient or a subject. As used herein, the term “patient” or “subject” refers to a living mammalian organism, such as a human, monkey, cow, sheep, pig, goat, dogs, cat, mouse, rat, guinea pig, or transgenic species thereof. In certain embodiments, the patient or subject is a primate. Non-limiting examples of human subjects are adults, juveniles, infants and fetuses. A patient or subject may have or may be at risk of having any condition or disease described herein.

Moreover, the type of biological matter varies. It can be cells, tissues and/or organs, as well as organisms. Moreover, it is contemplated in the context of cells and tissues that homogenous and heterogeneous cell populations may be the subject of embodiments of the invention.

As noted, methods and apparatuses of the invention may be applied to organisms. Protection and enhanced survivability of cells, tissues, and/or organs of the organism can be provided by methods and compositions of the invention. For example, protection or enhanced survivability can be provided for cells, tissues, or organs involving the heart, lung, kidney, liver, bone marrow, pancreas, skin, bone, vein, artery, cornea, blood, small intestine, large intestine, brain, spinal cord, smooth muscle, skeletal muscle, ovary, testis, uterus, and umbilical cord. Methods and apparatuses of the invention may also be used in in vivo biological matter. Biological matter that is contemplated for use with methods and apparatuses of the invention are limited only insofar as the comprise cells utilizing oxygen to produce energy.

Moreover, the invention can be employed with plants or parts of plants, including fruit, flowers, leaves, stems, seeds, cuttings. Plants can be agricultural, medicinal, or decorative. Methods and compositions used on plants may, for example, enhance the shelf life or pathogen resistance of the whole or part of the plant.

### III. Other Active Compounds

The present invention concerns methods, compositions and articles of manufacture involving one or more polychalcogenide compositions that can act on biological matter so as to produce a number of effects, including, but not limited to, enhancing or increasing survivability, reducing or preventing damage, preventing ischemic damage, preventing aging or senescence, and/or a achieve a variety of therapeutic applications discussed herein. It is contemplated that other compounds may be used in conjunction with polychalcogenide compositions of the invention in these contexts.

In some embodiments, the agent is an oxygen antagonist, which may act directly or indirectly. Oxygen antagonists are described in, for example, U.S. Patent Publns. 2005/0053912, 2005/0170019, 2005/0147692, 2005/0136125, 2008/0085329, and 2007/0078113, and U.S. patent application Ser. No. 11/738,294, all of which are hereby incorporated by reference in their entireties, each of which is incorporated herein by reference in its entirety. In some embodiments, the agent is a protective metabolic agent. Metabolism is generally understood as referring to chemical processes (in a cell or organism) that are required for life, including anabolism and catabolism. Protective metabolic agents are described in the above-noted references as well.

Another compound that may be used in conjunction with a polychalcogenide composition is carbon monoxide (CO). In certain embodiments, a polychalcogenide composition and CO are administered to induce stasis in biological material.

Reducing agents may be employed, in certain embodiments, along with a polychalcogenide composition. Reducing agents are well-known in the art. In certain embodiments, the reducing agent compound is dimethylsulfoxide, dimethylsulfide, methylmercaptan, mercaptoethanol, thiocyanate, hydrogen cyanide, or CS₂. Compounds on the order of the size of these molecules are also contemplated (that is, within about 50% of their molecular weights).

Additional compounds that may be employed, in certain embodiments, include, e.g., structures identified by CAS number in U.S. Patent Publn. 2007/0078113, incorporated herein by reference, many of which are readily available and are known to those of skill in the art. Further compounds that are contemplated as useful for methods of the invention include those with the chemical structure of Formulas I or IV, as disclosed in U.S. Patent Publn. 2007/0078113, which is hereby incorporated by reference.

In certain embodiments, hydrogen sulfide (H₂S) is employed with a polychalcogenide composition of the present invention. Hydrogen sulfide and its effects are described, for example, in U.S. Patent Publns. 2005/0170019, 2007/0078113 and U.S. patent application Ser. No. 11/738,294, each of which is incorporated herein by reference in its entirety. Methods of detecting and quantifying H₂S are described in, e.g., Hyspler et al., 2002, incorporated herein by reference in its entirety, and the references noted in this paragraph. The present invention also concerns the use of compounds and agents that can yield H₂S under certain conditions, such as upon exposure, or soon thereafter, to biological matter. It is contemplated that such precursors yield H₂S upon one or more enzymatic or chemical reactions. Indeed, administration of a polychalcogenide composition of the present invention to a subject may yield H₂S in vivo, for example.

Selectively targeting mitochondria is considered an embodiment of the invention in some aspects so as to enhance activity. Mitochondrial targeting agents may therefore be employed in certain embodiments, and these agents are known in the art. Certain agents are described in U.S. Patent Publn. 2007/0078113, incorporated herein by reference in its entirety.

Finally, any one or more compounds described in any of the following applications, each of which is incorporated herein by reference, may be employed with one or more polychalcogenide compositions of the present invention for any embodiment described herein: U.S. Patent Publns. 2005/0053912, 2005/0170019, 2005/0147692, 2005/0136125, 2008/0085329 and 2007/0078113 and U.S. patent application Ser. Nos. 11/837,536; 11/837,491; 11/837,539; 11/738,294; 11/864,355; 12/016,886; and 60/946,065.

### IV. Hypoxia and Anoxia

In certain embodiments, methods of the invention are carried out under hypoxic or anoxic conditions. Biological matter may be subjected to such conditions locally (i.e., to a certain part of the biological matter), or more typically, the entire biological matter may be exposed to such conditions.

Hypoxia is a common natural stress and several well conserved responses exist that facilitate cellular adaptation to hypoxic environments. To compensate for the decrease in the capacity for aerobic energy production in hypoxia, the cell must either increase anaerobic energy production or decrease energy demand (Hochachka et al., 1996). Examples of both of these responses are common in metazoans and the particular response used depends, in general, on the amount of oxygen available to the cell. Hypoxia and anoxia are described in U.S. Patent Publns. 2005/0170019, 2005/0147692, 2005/0136125, 2008/0085329 and 2007/0078113, each of which is incorporated herein by reference in its entirety.

“Hypoxia” occurs when the normal physiologic levels of oxygen are not supplied to a cell or tissue. “Normoxia” refers to normal physiologic levels of oxygen for the particular cell type, cell state or tissue in question. “Anoxia” is the absence of oxygen. “Hypoxic conditions” are those leading to cellular hypoxia. These conditions depend on cell type, and on the specific architecture or position of a cell within a tissue or organ, as well as the metabolic status of the cell. For purposes of the present invention, hypoxic conditions include conditions in which oxygen concentration is at or less than normal atmospheric conditions, that is less than 20.8, 20, 19, 18, 17, 16, 15, 14, 13, 12, 11, 10, 9, 8, 7, 6, 5, 4, 3, 2, 1, 0.5, 0%, or any range derivable therein; alternatively, these numbers could represent the percent of atmosphere at 1 atmosphere of pressure (101.3 kPa). An oxygen concentration of zero percent defines anoxic conditions. Thus, hypoxic conditions include anoxic conditions, although in some embodiments, hypoxic conditions of not less than 0.5% are implemented. As used herein, “normoxic conditions” constitute oxygen concentrations of around 20.8% or higher. Standard methods of achieving hypoxia or anoxia in biological matter are well established and include, for example, using environmental chambers that rely on chemical catalysts to remove oxygen from the chamber. See also U.S. Patent Publns. 2005/0053912, 2005/0170019 and 2007/0078113, each of which is incorporated herein by reference in its entirety.

It is contemplated that methods of the invention may use a combination of exposure to a polychalcogenide composition and alteration of oxygen concentrations compared to room air. Moreover, the oxygen concentration of the environment containing biological matter can be about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%, or any range derivable therein. Moreover, it is contemplated that a change in concentration can be any of the above percentages or ranges, in terms of a decrease or increase compared to room air or to a controlled environment.

### V. Stasis

In “stasis” or “suspended animation,” a cell, tissue or organ, or organism (collectively referred to as “biological material”) is living, but cellular functions necessary for cell division, developmental progression, and/or metabolic state are slowed or even stopped. Certain compounds, such as compounds that compete with oxygen, and certain conditions (e.g., hypoxic or anoxic conditions and hypothermia) have been shown to induce reversible stasis in animals. Stasis is described in, e.g., U.S. Patent Publns. 2005/0053912, 2005/0170019, 2005/0147692, 2005/0136125, 2008/0085329, 2007/0078113, and U.S. patent application Ser. No. 11/738,294, all of which are hereby incorporated in their entireties.

In certain embodiments, a polychalcogenide composition can be employed to induce and/or maintain stasis in biological matter: this method may be performed alone, or in combination with any other method described herein. Other patent applications discuss induction of stasis and compounds and/or conditions that can be used in methods and compositions of the present invention, such as U.S. Patent Publns. 2005/0053912, 2005/0170019, 2005/0147692, 2005/0136125, 2008/0085329, 2007/0078113, and U.S. patent application Ser. No. 11/738,294, all of which are hereby incorporated in their entireties. Any of the compounds or conditions described in these references can be used in combination with any polychalcogenide composition described herein to form compositions of the invention and to implement methods of the invention with respect to stasis or any other condition described herein.

Stasis is desirable in a number of contexts. Stasis can be used as a method of preservation by itself, or it may be induced as part of a cryopreservation regimen. Biological materials may be preserved for research use, for transportation, for transplantation, for therapeutic treatment (such as ex vivo therapy), and to prevent the onset of trauma, for example. Stasis with respect to entire organisms have similar uses. For instance, transportation of organisms could be facilitated if they had entered stasis. This might reduce physical and physiological damage to the organism by reducing or eliminating stress or physical injury. Stasis may be beneficial by decreasing the need of the biological material for oxygen and, therefore, bloodflow. It may extend the period of time that biological material can be isolated from a life-sustaining environment and exposed to a death-inducing environment.

Various compounds useful for inducing stasis may be initially evaluated using a variety of different tests. Stasis can be measured by a number of ways, including by quantifying the amount of oxygen consumed by a biological sample, the amount of carbon dioxide produced by the sample (indirect measurement of cellular respiration), or characterizing motility. CO₂ production, as described in, e.g., U.S. Patent Publn. 2007/0078113, incorporated herein by reference in its entirety, is a direct marker of cellular respiration related to metabolism of an organism.

### VI. Other Therapeutic or Preventative Applications

A. Trauma

In certain embodiments, the present invention may find use in the treatment of patients who are undergoing, or who are susceptible to trauma. Trauma may be caused by external insults, such as burns, wounds, amputations, gunshot wounds, or surgical trauma, internal insults, such as stroke or heart attack that result in the acute reduction in circulation, or reductions in circulation due to non-invasive stress, such as exposure to cold or radiation. On a cellular level, trauma often results in exposure of cells, tissues and/or organs to hypoxia, thereby resulting in induction of programmed cell death, or “apoptosis.” Systemically, trauma leads to the induction of a series of biochemical processes, such as clotting, inflammation, hypotension, and may give rise to shock, which if it persists may lead to organ dysfunction, irreversible cell damage and death. Biological processes are designed to defend the body against traumatic insult; however they may lead to a sequence of events that proves harmful and, in some instances, fatal. Therefore, the present invention contemplates the protection of tissues, organs, limbs and even whole organisms from the detrimental effects of trauma. See, e.g., U.S. Patent Publn. 2007/0078113, incorporated herein by reference in its entirety, for additional discussion and examples of trauma that may be treated using polychalcogenide compositions of the present invention.

The present invention also contemplates methods for inducing tissue regeneration and wound healing by prevention/delay of biological processes that may result in delayed wound healing and tissue regeneration. In addition to wound healing and hemorrhagic shock, methods of the invention can be implemented to prevent or treat trauma such as cardiac arrest or stroke. The invention has particular importance with respect to the risk of trauma from emergency surgical procedures, such as thoractomy, laparotomy, and splenic transection.

1. Wound Healing

In many instances, wounds and tissue damage are intractable or take excessive periods of time to heal. Examples are chronic open wounds (diabetic foot ulcers and stage 3 & 4 pressure ulcers), acute and traumatic wounds, flaps and grafts, and subacute wounds (i.e., dehisced incisions). This may also apply to other tissue damage, for example burns and lung damage from smoke/hot air inhalation.

In certain embodiments, methods of providing a polychalcogenide composition to biological matter is contemplated for the purpose of wound healing (that is, treating a wound). Previous experiments show hibernation to be protective against injury (e.g., pin's in brains), therefore it may have healing effects. Consequently, this technology may be useful in the control of wound healing processes, by bringing the tissue into a more metabolically controlled environment. Thus, in certain embodiments, biological matter may be induced into and/or kept in stasis following the provision of a polychalcogenide composition such that the would may be treated.

2. Hematologic Shock (Hemorrhagic Shock)

Shock is a life-threatening condition that progresses rapidly when interventions are delayed. Shock is a state in which adequate perfusion to sustain the physiologic needs of organ tissues is not present. This is a condition of profound haemodynamic and metabolic disturbance characterized by failure of the circulatory system to maintain adequate perfusion of vital organs. It may result from inadequate blood volume (hypovolaemic shock), inadequate cardiac function (cardiogenic shock) or inadequate vasomotor tone, also referred to as distributive shock (neurogenic shock, septic shock, anaphylactic shock). This often results in rapid mortality of the patient. Many conditions, including sepsis, blood loss, impaired autoregulation, and loss of autonomic tone, may produce shock or shocklike states. The present invention is anticipated to prevent detrimental effects of all the above states of shock, and sustain the life of the biological matter undergoing such shock.

In hemorrhagic shock, blood loss exceeds the body's ability to compensate and provide adequate tissue perfusion and oxygenation. This is frequently due to trauma, but may also be caused by spontaneous hemorrhage (e.g., gastrointestinal bleeding, childbirth), surgery, and other causes. Most frequently, clinical hemorrhagic shock is caused by an acute bleeding episode with a discrete precipitating event. Less commonly, hemorrhagic shock may be seen in chronic conditions with subacute blood loss. Diagnosis and management of the underlying hemorrhage must be performed rapidly and concurrently with management of shock. There are two major stages of shock: early compensation stage and progressive stage. It is contemplated that embodiments of the invention may be applied to patients in either or both stages.

Methods of treating hemorrhagic shock in a subject, wherein the subject is provided with a polychalcogenide composition, are contemplated by the present invention. In certain embodiments, the invention concerns inducing stasis in a patient, using a polychalcogenide composition to preserve the patient's vital organs and life. Stabilizing the patient in the first hour after injury is a major goal, and transport to a critical care facility (e.g., emergency room, surgery, etc.) where the injury can be properly addressed. Thus, it would be ideal to treat a patient with a polychalcogenide composition, such as by administering an effective amount of the composition to maintain the patient in stasis, to allow for this and to address immediate concerns such as source of shock, replenish blood loss, and reestablish homeostasis. While this will vary significantly, in most cases, the amount of time stasis will be maintained is between about 6 and about 72 hours after injury, but any timeframe discussed herein may be employed. In certain embodiments, a polychalcogenide composition and H₂S are administered to a subject to treat shock, as described in U.S. patent application Ser. No. 11/738,294, which is incorporated herein by reference in its entirety. See also U.S. Patent Publns. 2005/0170019 and 2007/0078113, each of which is incorporated herein in its entirety.

B. Hypothermia

In another embodiment, the inventors propose use of the present invention to treat people with extreme hypothermia. Mild hypothermia comprises achievement of a core body temperature of approximately between 0.1-5° C. below the normal core body temperature of the mammal. The normal core body temperature of a mammal is usually between 35-38° C. Moderate hypothermia comprises achievement of a core body temperature of approximately between 5-15° C. below the normal core body temperature of the mammal. Profound hypothermia comprises achievement of a core body temperature of approximately between 15-37° C. below the normal core body temperature of the mammal. Therapeutic benefits of hypothermia are described in, e.g., U.S. Patent Publn. 2007/0078113, incorporated herein by reference in its entirety.

Thus, certain embodiments contemplate providing a polychalcogenide composition to a subject in an effective amount to induce hypothermia in the subject. Methods and compositions of the present invention may, for example, be combined with invasive methods or medical devices known in the art to induce therapeutic hypothermia in mammals or humans. Such methods and devices are well-known in the art. See, e.g., U.S. Patent Publn. 2007/0078113, incorporated herein by reference in its entirety.

C. Hyperthermia

Under certain conditions, which can result from genetic, infectious, drug, or environmental causes, patients lose homeostatic temperature regulation resulting in severe uncontrollable fever (hyperthermia). This can result in mortality or long-term morbidity, especially brain damage, if not controlled properly. Administration of a polychalcogide composition may be used to control whole body temperature in certain states of hyperthermia in order to, for example, address the source of the fever. This may involve administration of a polychalcogenide composition through inhalation or perfused into the blood supply to induce a hibernation state. This can be combined with whole-body temperature regulation (ice bath/blanket/cooling system). It would be useful to have the patient undergo treatment for a time period sufficient to address the fever.

D. Cardioplegia and Coronary Heart Disease

In certain embodiments, polychalcogenide compositions may find use in solutions for the treatment of coronary heart disease (CHD) including a use for cardioplegia for coronary artery bypass surgery (CABG). CHD and CABG are well-known conditions.

Under certain conditions, such as those described in, e.g., U.S. Patent Publn. 2007/0078113, incorporated herein by reference in its entirety, it may be desirable to induce cardioplegia. Accordingly, the present invention provides methods, compounds and compositions for inducing cardioplegia that will, for example, provide greater protection to the heart during bypass surgery. The heart may be exposed to a polychalcogenide composition in such methods. In certain embodiments, the present invention provides a cardioplegic solution comprising a polychalcogenide composition dissolved in solution or bubbled as a gas in the solution. In certain embodiments, preventing or treating reperfusion injury associated with cardiac bypass surgery is contemplated. Reperfusion injury is described in, e.g., U.S. Patent Publn. 2007/0078113, incorporated herein by reference in its entirety. Bypass surgery typically last for 3-6 hours, however, complications and multiple vessel CABG can extend the duration to 12 hours or longer. A polychalcogenide composition may be provided to the heart, for example, for this amount of time. In certain embodiments, it is contemplated that the heart would be kept in stasis during the surgery. Methods of preparing and administering cardioplegic solutions are well-known in the art. See, e.g., U.S. Patent Publn. 2007/0078113, incorporated herein by reference in its entirety.

E. Reducing Damage from Cancer Therapy

Recent studies suggest that transient and reversible lowering of the core body temperature, or “hypothermia,” may lead to improvements in the fight against cancer. Based on this and other published work, the inventor proposes a further reduction in core temperature will provide benefit to cancer patients. Thus, the present invention contemplates the use of polychalcogenide compositions to protect normal tissues of a cancer patient, thereby reducing the potential impact of chemo- or radiotherapy on those tissues. It also permits the use of higher doses of chemo- and radiotherapy than tolerated without polychalcogenide composition exposure, thereby increasing the anti-cancer effects of these treatments.

Treatment of virtually any hyperproliferative disorder, including benign and malignant neoplasias, non-neoplastic hyperproliferative conditions, pre-neoplastic conditions, and precancerous lesions, is contemplated. Such disorders include restenosis, cancer, multi-drug resistant cancer, primary psoriasis and metastatic tumors, angiogenesis, rheumatoid arthritis, inflammatory bowel disease, psoriasis, eczema, and secondary cataracts, as well as oral hairy leukoplasia, bronchial dysplasia, carcinomas in situ, and intraepithelial hyperplasia. In particular, the present invention is directed at the treatment of human cancers including cancers of the prostate, lung, brain, skin, liver, breast, lymphoid system, stomach, testicles, ovaries, pancreas, bone, bone marrow, gastrointestine, head and neck, cervix, esophagus, eye, gall bladder, kidney, adrenal glands, heart, colon and blood. Cancers involving epithelial and endothelial cells are also contemplated for treatment.

Polychalcogenide compositions may be combined with anti-cancer agents, as described in U.S. Patent Publn. 2007/0078113, incorporated herein by reference in its entirety. An “anti-cancer” agent is capable of negatively affecting cancer in a subject, for example, by killing, inducing apoptosis in, or reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. Methods of co-administration with anti-cancer agents and therapies are described in e.g., U.S. Patent Publn. 2007/0078113, incorporated herein by reference in its entirety. Various surgical interventions, chemotherapies, radiotherapies and/or immunotherapies that may be employed in certain embodiments are described in, e.g., U.S. Patent Publn. 2007/0078113, incorporated herein by reference in its entirety.

Administration of a polychalcogenide composition of the present invention to a patient will follow general protocols for the administration of chemotherapeutics, taking into account the toxicity, if any, of the compound. It is expected that the treatment cycles would be repeated as necessary. Any timeframe of polychalcogenide composition administration discussed herein may be employed.

F. Neurodegeneration and Aging

The present invention may be used to treat neurodegenerative diseases. Neurodegenerative diseases are characterized by degeneration of neuronal tissue, and are often accompanied by loss of memory, loss of motor function, and dementia. Neurodegenerative diseases include Parkinson's disease; primary neurodegenerative disease; Huntington's Chorea; stroke and other hypoxic or ischemic processes; neurotrauma; metabolically induced neurological damage; sequelae from cerebral seizures; hemorrhagic shock; secondary neurodegenerative disease (metabolic or toxic); Alzheimer's disease, other memory disorders; or vascular dementia, multi-infarct dementia, Lewy body dementia, or neurodegenerative dementia.

Furthermore, reduced metabolic activity overall has been shown to correlate with health in aged animals and humans. Therefore, the present invention would also be useful to suppress overall metabolic function to increase longevity and health in old age. It is contemplated that this type of treatment would likely involve administration of a polychalcogenide composition at night, during sleep, for period of approximately 6 to 10 hours each day, although any timeframe discussed herein may be employed.

Furthermore, aging itself may be thoroughly or completely inhibited for the period of time when the biological matter is exposed to a polychalcogenide composition. Thus the present invention may inhibit aging of biological material, with respect to extending the amount of time the biological material would normally survive and/or with respect to progression from one developmental stage of life to another.

G. Blood Disease

A number of blood diseases and conditions may be addressed using polychalcogenide compositions and methods involving the same. These diseases include, but are not limited to, thalassemia and sickle cell anemia. These conditions are described in, e.g., U.S. Patent Publn. 2007/0078113, incorporated herein by reference in its entirety.

### VII. Preservation Applications

The present invention can be used to preserve or store a variety of biological matter, including cells, tissues, organs, and organisms for transport and/or storage purposes. In certain embodiments, the biological matter is preserved so as to prevent damage from adverse conditions. It may be desirable to preserve biological matter, so as to prevent as much as possible damage to the matter from perishing or decomposing. It is contemplated that polychalcogenide compositions may be used to preserve biological matter for any significant period of time or prepare the biological matter for such preservation. This may be in combination with other agents, such environmental changes in pressure and/or temperature. Preservation benefits and techniques are described in, e.g., U.S. Patent Publns. 2005/0170019 and 2007/0078113, each of which is incorporated herein by reference in its entirety.

As discussed above, a variety of cells are contemplated for use with the present invention. It is contemplated that such cells can be preserved in methods, apparatuses, and compositions of the invention. Particular types of cells include, for example, platelets, as described in U.S. Patent Publn. 2007/0078113, incorporated herein by reference in its entirety. Suspension, storage, assays for platelet function, and equipment pertaining to each of these aspects are known in the art. See, e.g., U.S. Pat. Nos. 4,828,976; 4,447,415; 6,790,603; 5,470,738; and 5,231,025; and U.S. Patent Publn. 2007/0078113, each of which is incorporated herein by reference in its entirety. The cell type may be any type described herein, such as tumor cells and epithelial cells.

The present invention may be extended to protecting cells in culture, which might otherwise die or be induced into apoptosis. Cells may be exposed to a polychalcogenide composition prior to and/or while in culture. Cell culture techniques are well-known in the art. See, e.g., U.S. Patent Publn. 2007/0078113, Davis (1994) and U.S. Pat. Nos. 5,580,781 and 6,057,148, each of which is incorporated by reference in its entirety.

In the context of preservation with respect to transplanting biological matter, it is envisioned that donated and recipient biological matter, such as tissues or organs, will be treated pre-transplantation with a polychalcogenide composition in an effort to inhibit biological processes such as inflammation, apoptosis and other wound healing/post-transplantation events that cause damage to, for example, engrafted tissues.

A. Preservation Agents

A variety of preservation solutions have been disclosed in which the organ is surrounded or perfused with the preservation solution while it is transported. Any of these agents may be employed in methods and compositions of the present invention. Such agents are described in, e.g., U.S. Patent Publn. 2007/0078113, incorporated herein by reference in its entirety. It is contemplated that any agent or solution used with a biological sample that is living and that will be used as a living material will be pharmaceutically acceptable or pharmacologically acceptable.

B. Preservation Apparatuses and Applications

Systems and containers for transporting organs and tissues have been developed through the years as well as methods relating thereto. See, e.g., U.S. Patent Publn. 2007/0078113, Kuroda et al., 1988 and U.S. Pat. Nos. 4,292,817; 4,473,637; 4,745,759; 5,434,045; 4,723,974; 6,490,880; 6,100,082; 6,046,046; 6,054,261; 5,326,706; 5,285,657; 5,157,930; 4,951,482; 4,502,295; 4,186,565; 6,490,880; 6,046,046; 5,476,763; 5,285,657; 3,995,444; 3,881,990 and 3,777,507, each of which is incorporated herein by reference in its entirety. Any polychalcogenide composition described in this application is contemplated for use with preservation methods, apparatuses, and/or applications.

### VIII. Diagnostic Applications

Sulfites are produced by all cells in the body during normal metabolism of sulfur containing amino acids. Sulfite oxidase removes and thus regulates the levels of sulfites. For solid tumors in hypoxic conditions, for example, sulfites may be produced at higher levels to provide local protective state to the tumor cells through the reduction of metabolic state as well as the inhibition of immune surveillance. Therefore, it would be beneficial to measure sulfite levels and incorporate this as part of diagnosis for several disease states such as solid tumors. Furthermore, it may be useful to follow this using an imaging or other monitoring process. It is possible to measure sulfite levels in serum to get a total sulfite level using current technology (e.g., HPLC). Any measuring methods associated with sulfite and/or sulfite oxidase detection may be used in any embodiment described herein.

### IX. Screening Applications

In still further embodiments, the present invention provides methods for identifying polychalcogenide compositions, compounds, and/or salts that act in a like fashion regarding enhancing survivability of and/or protecting biological matter. In some cases, the composition being sought or tested works like a polychalcogenide composition in reducing core body temperature or preserving viability in hypoxic or anoxic environments that would otherwise kill the biological matter if it were not for the presence of the polychalcogenide composition. These assays may comprise random screening of large libraries of candidate substances; alternatively, the assays may be used to focus on particular classes of composition selected with an eye towards attributes that are believed to make them more likely to act as polychalcogenide compositions of the invention. In particular embodiments, this involves providing a candidate polychalcogenide composition; (a) admixing the candidate polychalcogenide composition with a biological matter; (b) measuring one or more cellular responses characteristic of polychalcogenide composition treatment; and (c) comparing the one or more responses with the biological matter in the absence of the candidate polychalcogenide composition. Assays may be conducted with isolated cells, tissues/organs, or intact organisms.

The invention provides methods for screening for such candidates, not solely methods of finding them. However, it will also be understand that a candidate composition may be identified as an effective polychalcogenide composition according to one or more assays, meaning that the candidate polychalcogenide composition appears to have some ability to act as a protective agent in a biological matter. Any methods discussed herein may be combined with a screening method of the present invention.

### X. Modes of Administration and Pharmaceutical Compositions

An effective amount of a pharmaceutical composition comprising a polychalcogenide composition is generally defined as that amount sufficient to detectably ameliorate, reduce, minimize or limit the extent of the condition of interest. More rigorous definitions may apply, including elimination, eradication or cure of disease. A pharmaceutical composition is formulated such that it is pharmaceutically acceptable.

The routes of administration of a polychalcogenide compound, polychalcogenide composition, polychalcogenide salt, compound comprising a Ch-(Ch)ₙ-Ch group, or a pharmaceutical composition comprising a polychalcogenide compound, polychalcogenide composition, polychalcogenide salt, or compound comprising a Ch-(Ch)ₙ-Ch group will vary, naturally, with the location and nature of the condition to be treated, and include, e.g., inhalation, intradermal, transdermal, parenteral, intravenous, intra-arterial, intramuscular, intranasal, subcutaneous, percutaneous, intratracheal, intraperitoneal, intratumoral, perfusion, lavage, direct injection, and oral administration and formulation. Compounds may be administered as medical gases by inhalation or intubation, as injectable liquids by intravascular, intravenous, intra-arterial, intracerobroventicular, intraperitoneal, subcutaneous administration, as topical liquids or gels, or in solid oral dosage forms.

Particular examples of injectable compositions and formulations, intravenous formulations, topical formulations, solid dosage forms, perfusion systems, catheters, gas delivery devices (wherein at least one polychalcogenide compound is in the form of a gas), other delivery devices and apparatuses that may be employed using a polychalcogenide composition of the present invention are described in, e.g., U.S. Patent Publn. 2007/0078113, incorporated herein by reference in its entirety.

The amounts may vary depending on the type of biological matter (cell type, tissue type, organism genus and species, etc.) and/or its size (weight, surface area, etc.). It will generally be the case that the larger the organism, the larger the dose. Therefore, an effective amount for a mouse will generally be lower than an effective amount for a rat, which will generally be lower than an effective amount for a dog, which will generally be lower than an effective amount for a human.

Similarly, the length of time of administration may vary depending on the type of biological matter (cell type, tissue type, organism genus and species, etc.) and/or its size (weight, surface area, etc.) and will depend in part upon dosage form and route of administration. Dosages and timeframes of administration are discussed herein.

In the case of transplant, the present invention may be used pre- and or post-operatively to render host or graft materials quiescent. In a specific embodiment, a surgical site may be injected or perfused with a formulation comprising a polychalcogenide composition. The perfusion may be continued post-surgery, for example, by leaving a catheter implanted at the site of the surgery.

“Pharmaceutically acceptable” means that which is useful in preparing a pharmaceutical composition that is generally safe, non-toxic and neither biologically nor otherwise undesirable and includes that which is acceptable for veterinary use as well as human pharmaceutical use. Polychalcogenide compositions may be comprised in pharmaceutically acceptable compositions, for example.

“Pharmaceutically acceptable salts” means salts of compounds or compositions of the present invention which are pharmaceutically acceptable, as defined above, and which possess the desired pharmacological activity. Polychalcogenide compounds of the present invention are contemplated in their pharmaceutically acceptable salt forms, and polychalcogenide compositions, polychalcogenide compounds, polychalcogenide salts, and compound comprising a Ch-(Ch)ₙ-Ch group may comprise pharmaceutically acceptable salts. Pharmaceutically acceptable salts include acid addition salts formed with inorganic acids such as hydrochloric acid, hydrobromic acid, sulfuric acid, nitric acid, phosphoric acid, and the like; or with organic acids such as 1,2-ethanedisulfonic acid, acetic acid, aliphatic mono- and dicarboxylic acids, lactic acid, laurylsulfuric acid, maleic acid, oxalic acid, tartaric acid, and the like. Pharmaceutically acceptable salts also include base addition salts which may be formed when acidic protons present are capable of reacting with inorganic or organic bases. Exemplary inorganic bases include sodium hydroxide, sodium carbonate, potassium hydroxide, aluminum hydroxide and calcium hydroxide. Exemplary organic bases include ethanolamine, diethanolamine, triethanolamine, tromethamine, N-methylglucamine and the like. Additional examples of pharmaceutically acceptable salts and their methods of preparation and use are presented in Handbook of Pharmaceutical Salts: Properties, Selection and Use (P. H. Stahl & C. G. Wermuth eds., Verlag Helvetica Chimica Acta, 2002), which is incorporated herein by reference.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions. In certain embodiments, pharmaceutical compositions of the present invention include one more excipients that are reducing agents (see: U.S. Pat. No. 6,586,404, incorporated herein by reference).

### XI. Combination Therapies

The compositions, compounds, salts and methods of the present invention may be used in the context of a number of therapeutic and diagnostic applications. In order to increase the effectiveness of a treatment with for example, polychalcogenide compositions, it may be desirable to combine these compositions with other agents effective in the treatment of those diseases and conditions (secondary therapy). For example, the treatment of stroke (antistroke treatment) typically involves an antiplatelet (e.g., aspirin, clopidogrel, dipyridamole, ticlopidine), an anticoagulant (e.g., heparin, warfarin), or a thrombolytic (e.g., tissue plasminogen activator).

Various combinations may be employed; for example a polychalcogenide composition is “A” and the secondary therapy is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of polychalcogenide compositions of the present invention to biological matter will follow general protocols for the administration of that particular secondary therapy, taking into account the toxicity, if any, of the polychalcogenide composition treatment. It is expected that the treatment cycles would be repeated as necessary. It also is contemplated that various standard therapies, as well as surgical intervention, may be applied in combination with the described therapies.

### XII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Preparation of Sodium Polysulfide Oil

The following method is taken from the literature (Steudel, 2003a, b). Sodium polysulfide oil was synthesized by adding 2 parts sodium sulfide nonahydrate (Aldrich product no. 431648) with one part elemental sulfur (S₈) (Fisher catalog no. S594-500; material is preferentially soluble in carbon tetrachloride); resulting solution is 800 mg/ml sodium polysulfide oil solution. One part of this solution is mixed with 9 parts 20 mM HCl; the resulting solution is 80 mg/ml sodium polysulfide oil. The resulting product was a mixture of Na₂Sₙ compounds, wherein n ranges from 2 to 35, including both integers and non-integers. Sodium polysulfide oil comprised an average of 5.5 sulfur atoms.

### Example 2

### Administration of Sodium Polysulfide Oil to Pigs Results in a Reduction of Tachycardia with Minimal Effect on Blood Pressure

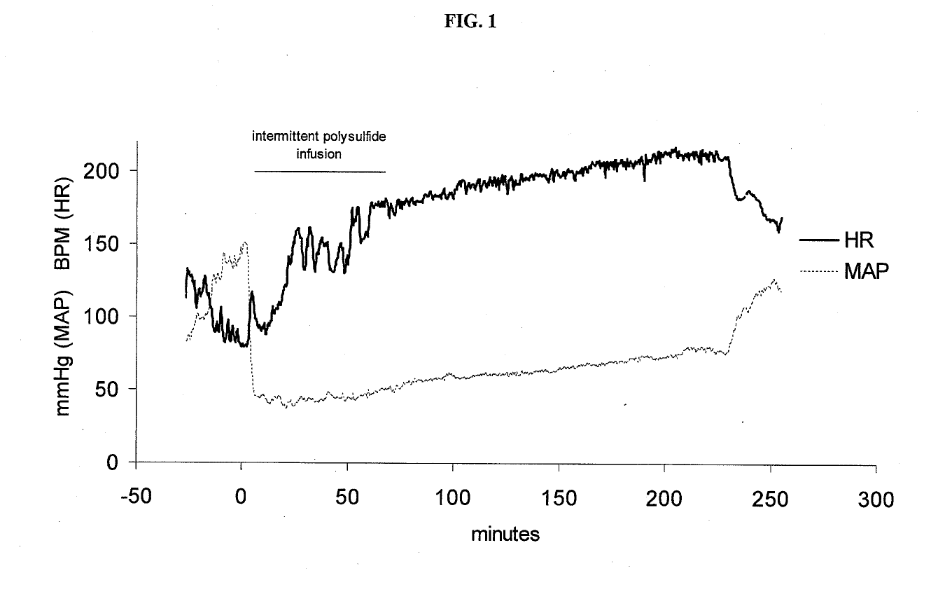

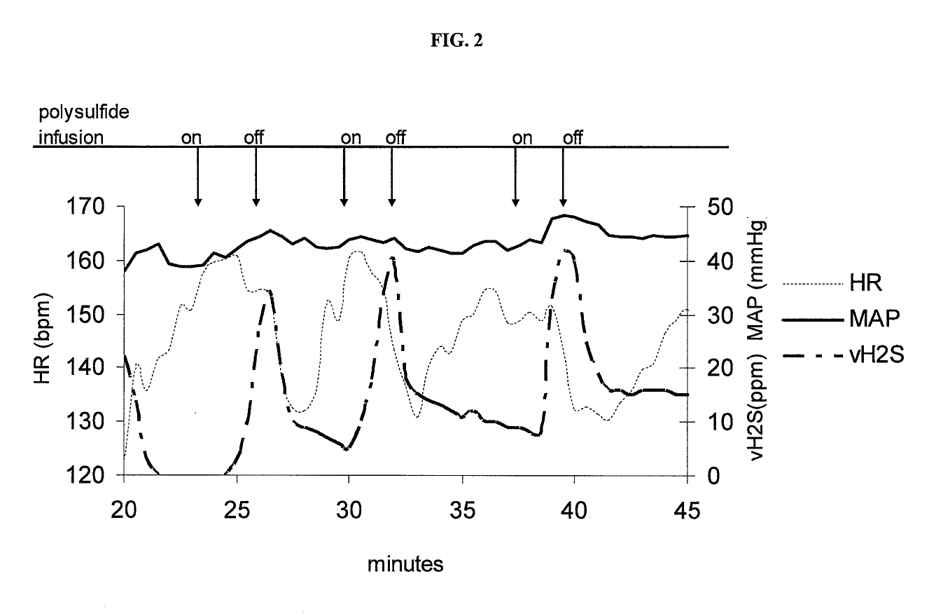

Three separate pigs were tested with the following protocol, the results of which can be seen in FIG. 1 and FIG. 2. FIG. 1 shows the results of the entire timespan of one experiment while FIG. 2 focuses on minutes 20-45 of the same experiment.

A pig was dosed six times with a sodium polysulfide oil as prepared in Example 1 (80 mg/ml). Infusion took place through a femoral vein catheter using an infusion pump. The first dose, 60 mg/kg/hr delivered with a Harvard syringe pump, administered after 400 out of 800 ml of blood was removed (approximately 10 minutes after blood removal began), caused 27 ppm of hydrogen sulfide (H₂S) to be exhaled before the infusion was stopped (total infusion time was 5 minutes). The second dose of 60 mg/kg/hr was begun approximately 5 minutes later. After one minute the pig exhaled 34 ppm H₂S and the heart rate (HR) fell from 160 beats per minute (bpm) to 133 bpm. At this time the infusion rate was reduced to 20 mg/kg/hr; the exhaled H₂S decreased to 5 ppm and the HR increased to 160 bpm. Five minutes later the dose was increased to 60 mg/kg/hr; the exhaled H₂S reached 40 ppm and the HR decreased to 130 bpm within 1 minute. The infusion rate was reduced to 20 mg/kg/hr; the exhaled H₂S decreased to 8 ppm and the HR increased to 155 bpm. After 7 minutes the dose was increased to 60 mg/kg/hr; the exhaled H₂S increased to 44 ppm and the heart rate decreased to 131 bpm. The infusion rate was reduced to 20 mg/kg/hr; exhaled H₂S decreased to 15 ppm and HR increased to 150 bpm. After 6 minutes, the dose was increased to 60 mg/kg/hr; the exhaled H₂S increased to 27 ppm and HR decreased to 130 bpm. At this time blood removal stopped. Sodium polysulfide oil infusion was also stopped. Exhaled H₂S decreased to 0 ppm and HR increased to 175 bpm. Three minutes after the end of blood removal, sodium polysulfide oil infusion was restarted at 20 mg/kg/hr. After 15 seconds, exhaled H₂₅ increased to 27 ppm and HR decreased to 163 bpm. After 2 minutes of 20 mg/kg/hr, exhaled H₂S decrease to 0 ppm and HR increased to 175. Infusion rate was increased to 60 mg/kg/hr; within 1 minute, exhaled H₂S increased to 28 ppm and HR decreased to 150 bpm. The infusion rate was decreased to 20 mg/kg/hr, exhaled H₂S decreased to 15 ppm and HR increased to 179. After 4 minutes, sodium polysulfide oil infusion was stopped.

A total of 5.6 ml of sodium polysulfide oil was administered (448 mg). Exhaled sulfide was monitored using a hand-held Thermo Gastech Innova gas monitor. Heart rate was monitored using an ADI instruments ECG monitor and DSI telemetry. Mean arterial blood pressure was monitored using DSI telemetry.

As demonstrated in FIG. 1 and FIG. 2, there were no significant changes in mean arterial blood pressure (MAP) during this time.

### Example 3

### Administration of Sodium Polysulfide Oil Protects Against Lethal Hemorrhage

To determine whether sodium polysulfide oil could be used to reduce morbidity and/or tissue damage associated with a clinically relevant acute injury model of ischemic hypoxia, rats were treated with the sodium polysulfide oil of Example 1 during controlled lethal hemorrhage. In this study, rats treated with sodium polysulfide oil survived lethal blood loss and fully recovered.

Rats were treated with sodium polysulfide oil during controlled lethal hemorrhage (60% blood loss). After surgical implantation of catheters and recovery, blood was removed from conscious animals over 40 minutes. A sodium polysulfide oil dosage of approx. 30 mg/kg/hr was infused during the last 20 minutes of the bleed and during the first 20 minutes after the bleed had stopped. Three hours after the end of the bleed, surviving animals were given one shed-blood volume of lactated ringers solution intravenously. Most (5/8) of the treated rats survived hemorrhage and recovered completely.

### Example 4

### Preparation of Sodium Polysulfide Oil in Combination with Human Serum Albumin and Glutathione

Following the procedure of Example 1, to a 15 mL polypropylene conical tube (Falcon) was added 1 gram of powdered yellow sulfur (elemental S₈, Fisher catalog no. S594-500; material is preferentially soluble in carbon tetrachloride) and 2 grams of powdered sodium sulfide nonahydrate (Aldrich product no. 431648). The tube was capped and shaken thoroughly to ensure thorough mixing. The tube was placed in a hot water bath that had been brought to boiling, and the tube was allowed to sit in the bath overnight with no additional heating (that is, the bath was boiling hot initially and was allowed to cool overnight). A viscous orange mixture resulted. This mixture was spun for 15 min at 1500 RPM, whereupon a precipitate containing elemental sulfur formed. The supernatant, which was a viscous orange-yellow oil was decanted. This supernatant was diluted 1:10 with 20 mM HCl (HCl added to oil) to form 80 mg/ml sodium polysulfide oil with a pH of about 9. This dilution step typically takes place just prior to use.

To prepare the glutathione solution (100 mg/ml), 0.5 g reduced glutathione (GSH, Sigma catalog no. G4251) or 0.25 g GSH plus 0.25 g oxidized glutathione (GSSG, MP Biomedicals catalog no. 151193) was suspended in 3.0 ml H₂O. The solution was brought to pH 7.0 with 1.5 ml 1N NaOH.

A syringe was used to draw up 2 mL of the 100 mg/ml glutathione solution. The same syringe was then used to draw up 4 ml of 250 mg/ml human serum albumin solution (Plasbumin® 25 from Talecris, catalog no. NDC 13533-684-16, 25% w/w solution of human serium albumin). The same syringe was then used to draw up 0.5 ml of the 80 mg/ml sodium polysulfide oil. The syringe and its contents (6.5 ml of sodium polysulfide oil/glutathione/albumin solution) were then mixed, and then allowed to sit at room temperature for 10 min with intermittent shaking before use.

### Example 5

### Administration of Sodium Polysulfide Oil in Combination with Human Serum Albumin and/or Glutathione: a Hemorrhagic Shock Study

Swine Model:

Catheters and a telemeter were placed surgically in a pig. The pig was recovered for 30 minutes until behavior and physiology were stable. The pig was conscious in a sling and was sedated with midazolam. Blood was removed from the pig in 20-60 minutes. Blood pressure was decreased to 40 mm Hg within 10 minutes.

If, after 36 ml/kg of blood was removed, (i) the heart rate was over 150 beats per minute; (ii) base excess was equal to or less than −15 mmol/L; and (iii) the T-wave was inverted on an electrocardiogram, blood removal was ended. If all three of the above criteria were not met, blood volume removed was increased to 39 ml/kg. If 2/3 of the above criteria were met, blood removal ended. If not, blood volume removed was increased to 42 ml/kg. If 1/3 of the criteria were met, blood removal ended. If not, blood volume removed was increased to 45 ml/kg. Blood removal ended at this time regardless of physiological criteria. Within 5 minutes after blood removal ended, control or test formulation was administered intravenously.

Ending physiological criteria:
1. Heart rate greater than 150 beats per minute
2. Base excess equal to or less than −15 mmol/L
3. Inverted T-wave on electrocardiogram

| | ml/kg | criteria |
|---|---|---|
| Blood removal ended at: | 36 | 3/3 |
| | 39 | 2/3 |
| | 42 | 1/3 |
| | 45 | 0/3 |

Pigs received 2 mg/kg of 80 mg/ml sodium polysulfide oil mixed with 2 volumes of 100 mg/ml glutathione and 4 volumes 250 mg/ml human serum albumin, as described in Example 4. In other words, pigs received the polysulfide composition as prepared in Example 4. For example: a 20 kg pig requires 40 mg sodium polysulfide oil, which is contained in 0.5 ml of the 80 mg/ml sodium polysulfide oil. Therefore, 2.0 ml of 100 mg/ml glutathione is added to 4.0 ml of 250 mg/ml human serum albumin. To this 6.0 ml, 0.5 ml of 80 mg/ml sodium polysulfide oil is added within 15 minutes of use. The entire 6.5 ml bolus is administered intravenously in approximately 20 seconds. Physiological responses to administration of test formulation: within 15 seconds, hydrogen sulfide gas was exhaled for up to 2 minutes and up to or over 200 ppm. Hydrogen sulfide gas was measured by a gas sensor. 30-120 seconds after administration, heart rate decreased 10-30% and mean arterial pressure rose 10-50%. Surviving pigs were resuscitated with shed blood 3 hours after blood removal.

### Results:

In one experiment, eighteen pigs were treated, with results as indicated:

| | | HSA + Sodium | HSA + GSH + Sodium | |
|---|---|---|---|---|
| | Control | Polysulfide Oil | Polysulfide Oil | HSA + GSH |
| Live | 0 | 4 | 2 | 0 |
| Dead | 6 | 5 | 0 | 1 |

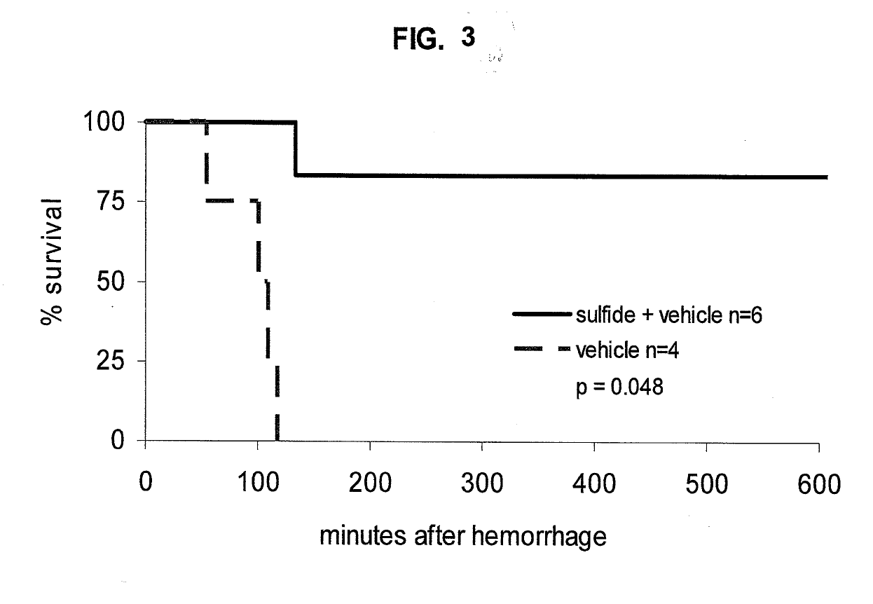

The results of a second experiment are shown in FIG. 3.

### Example 6

### Rats and Pits Exhale H2S after Elemental Sulfur Administration

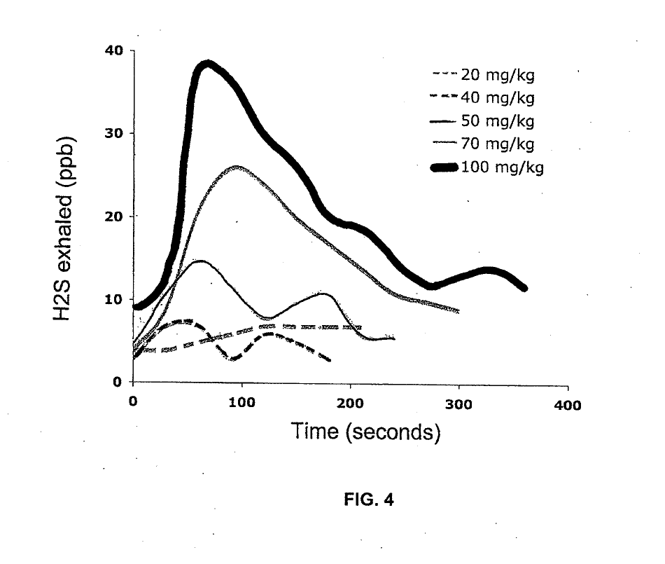

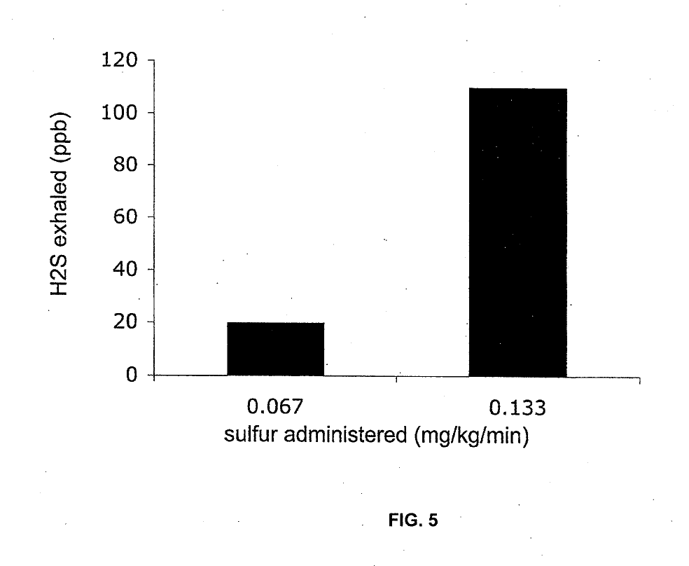

As shown in FIG. 4, rats administered colloidal elemental sulfur through a femoral vein catheter exhale H2S. The amount of colloidal sulfur administered was 2 mg/kg delivered as a 0.3 ml/kg solution. The graph indicates that rats are able to reduce elemental sulfur (sulfur in the 0 oxidation state) to H2S (sulfur in the −2 oxidation state) in a dose-dependent manner; the more elemental sulfur administered, the more H2S exhaled. Exhaled gas was measured using a gas sensor. FIG. 5 shows that pigs administered colloidal sulfur through a venous catheter as a constant infusion in the amounts indicated also exhale H2S.

### Example 7

### Pigs Exhale H2S after Sodium Polysulfide Oil Administration

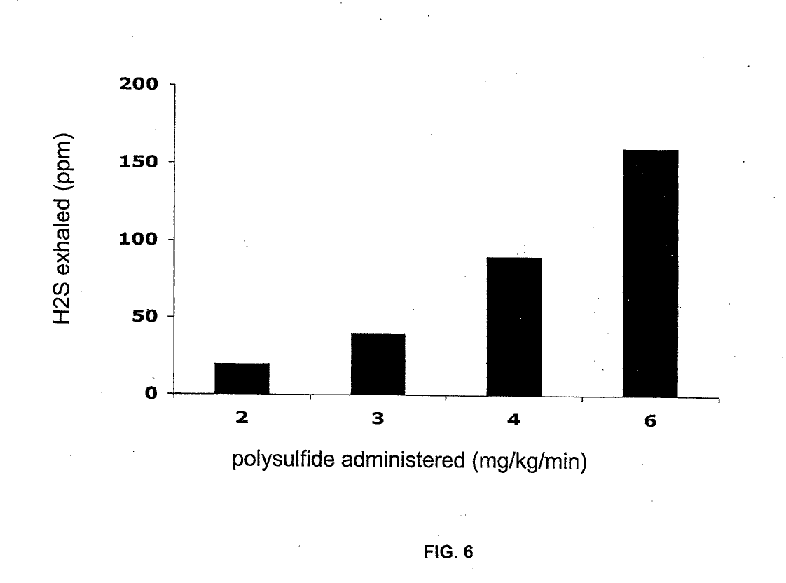

As shown in FIG. 6, pigs exhale H2S after sodium polysulfide oil administration through a venous catheter as a constant infusion in the amounts indicated. The graph indicates that pigs can reduce this polysulfide (with one or more sulfur atoms in approximately the −0.4 oxidation state) to H2S (sulfur in the −2.0 oxidation state) in a dose-dependent manner. Exhaled gas was measured using a gas sensor. The concentration of H2S shown on the y-axis of FIG. 6 is the value measured after 3 minutes of infusion.

### Example 8

### Survival of Pigs after Lethal Hemorrhage Using Compounds and Compositions of the Present Invention

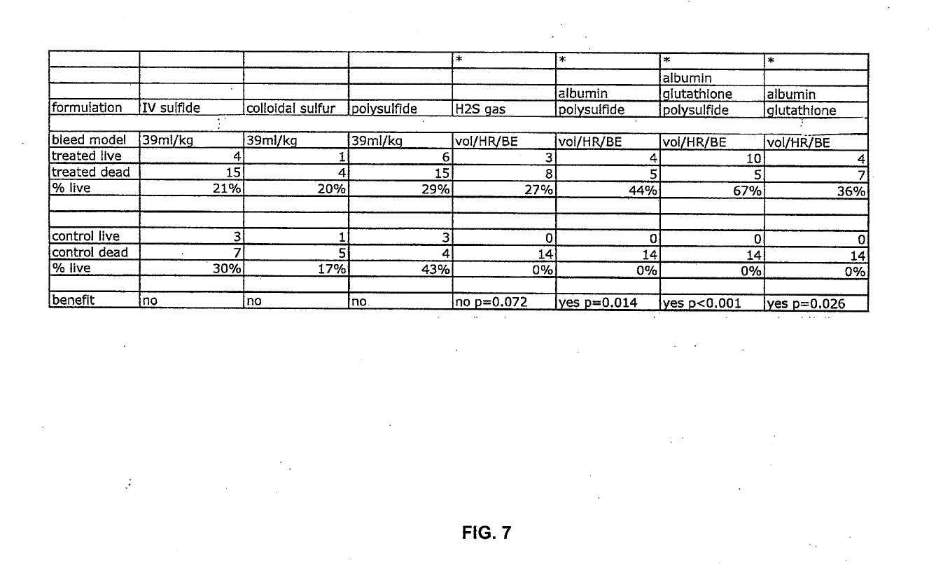

The table shown in FIG. 7 demonstrates that various compositions comprising multiple sulfur atoms can protect pigs from lethal hemorrhage.

Pigs were subjected to lethal hemorrhage (either 39 ml/kg or variable volume based on heart rate (HR), mean arterial pressure (MAP), and blood base excess (BE), as described in Example 5). If pigs survived 3 hours after end of hemorrhage, they were resuscitated with shed blood. All pigs that lived 3 hours and were resuscitated, survived the 24 monitoring period with no observable behavioral or functional defects, and were considered survivors in the table above. Formulations and bleed models are listed at the top of each column. The total numbers of treated and control animals that lived and died are listed in the rows. One animal was tested per day. Benefit was assayed by performing Fisher's exact two-sided analysis between treated and control groups.

All experiments were performed under the same conditions. Animals were housed individually. Food and water were provided ad libitum. All experiments conformed to the U.S. National Institutes of Health guidelines regulating the care and use of laboratory animals.

Test agent was provided intravenously at 0.3 ml/kg unless by gas, which was administered as described below, and animals received it at the end of the bleed. The amounts provided in each column of the table are as follows: IV sulfide: 1.0 mg/kg; elemental colloidal sulfur: 2 mg/kg; sodium polysulfide: 2 mg/kg; H2S gas: 3000 ppm at a rate wherein the one-way valve in the administration apparatus stayed shut such that external air did not enter the mask (typically about 40 L/min); albumin polysulfide is a composition comprising 15% human serum albumin (Talecris) and 2 mg/kg sodium polysulfide oil; albumin glutathione polysulfide is a composition comprising 15% human serum albumin (Talecris), 30 mg/ml glutathione, and 2 mg/kg sodium polysulfide oil; albumin glutathione is a solution comprising 15% human serum albumin (Talecris) and 30 mg/ml glutathione.

The control group for these studies includes 4 animals that received an equal volume of saline and 10 animals that received nothing at the end of the bleed. Comparisons between any two of these test groups was not statistically significant (p>0.05).

### Example 9

### Survival of Pigs after Lethal Hemorrhage and Treatment Using Polysulfide Compounds and Compositions

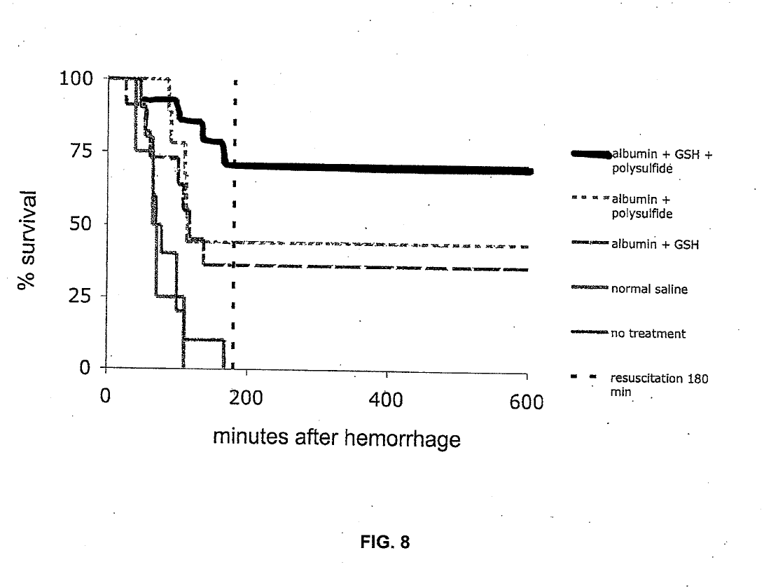

The Kaplan-Meier graph shown in FIG. 8 demonstrates that various sulfur-containing compositions can protect pigs from lethal hemorrhage. Amounts and routes of administration are provided in Example 8.

### Example 10

### Sodium Sulfide is Oxidized to a Polysulfide by Albumin

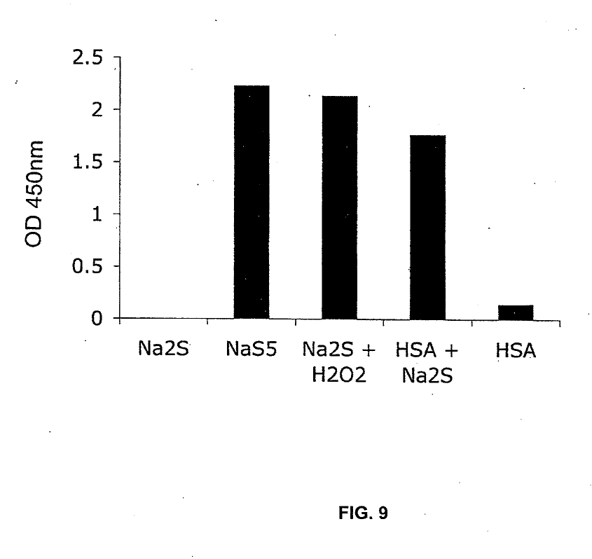

The graph in FIG. 9 shows that sulfide can be converted to polysulfide by human serum albumin. Sodium sulfide (Na₂S, 60 mM) is not yellow in color and does not absorb light at 450 nm. Sodium polysulfide (Na₂S₅, 60 mM) is yellow in color because of its absorbance of light of 450 nm. Sodium sulfide (60 mM) can be oxidized to polysulfide by addition of hydrogen peroxide (H₂O₂, 10 mM). Sodium sulfide (60 mM) can also be oxidized to polysulfide by human serum albumin (150 mg/ml). Human serum albumin (150 mg/ml) weakly absorbs at 450 nm.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods, and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 3,777,507
U.S. Pat. No. 3,881,990
U.S. Pat. No. 3,989,816
U.S. Pat. No. 3,995,444
U.S. Pat. No. 4,186,565
U.S. Pat. No. 4,292,817
U.S. Pat. No. 4,444,762
U.S. Pat. No. 4,447,415
U.S. Pat. No. 4,473,637
U.S. Pat. No. 4,502,295
U.S. Pat. No. 4,502,295
U.S. Pat. No. 4,559,258
U.S. Pat. No. 4,723,974
U.S. Pat. No. 4,745,759
U.S. Pat. No. 4,765,539
U.S. Pat. No. 4,790,327
U.S. Pat. No. 4,798,824
U.S. Pat. No. 4,828,976
U.S. Pat. No. 4,938,961
U.S. Pat. No. 4,951,482
U.S. Pat. No. 4,962,885
U.S. Pat. No. 5,066,578
U.S. Pat. No. 5,157,930
U.S. Pat. No. 5,217,860
U.S. Pat. No. 5,231,025
U.S. Pat. No. 5,285,657
U.S. Pat. No. 5,326,706
U.S. Pat. No. 5,370,989
U.S. Pat. No. 5,395,314
U.S. Pat. No. 5,399,363
U.S. Pat. No. 5,405,742
U.S. Pat. No. 5,434,045
U.S. Pat. No. 5,466,468
U.S. Pat. No. 5,470,738
U.S. Pat. No. 5,476,763
U.S. Pat. No. 5,543,158
U.S. Pat. No. 5,552,267
U.S. Pat. No. 5,569,579
U.S. Pat. No. 5,580,781
U.S. Pat. No. 5,599,659
U.S. Pat. No. 5,641,515
U.S. Pat. No. 5,693,462
U.S. Pat. No. 5,699,793
U.S. Pat. No. 5,719,174
U.S. Pat. No. 5,736,397
U.S. Pat. No. 5,739,169
U.S. Pat. No. 5,752,929
U.S. Pat. No. 5,801,005
U.S. Pat. No. 5,830,880
U.S. Pat. No. 5,846,945
U.S. Pat. No. 5,912,019
U.S. Pat. No. 5,950,619
U.S. Pat. No. 5,952,168
U.S. Pat. No. 5,954,047
U.S. Pat. No. 5,970,974
U.S. Pat. No. 5,988,162
U.S. Pat. No. 6,013,256
U.S. Pat. No. 6,046,046
U.S. Pat. No. 6,054,261
U.S. Pat. No. 6,057,148
U.S. Pat. No. 6,100,082
U.S. Pat. No. 6,123,524
U.S. Pat. No. 6,187,529
U.S. Pat. No. 6,195,887
U.S. Pat. No. 6,213,723
U.S. Pat. No. 6,213,738
U.S. Pat. No. 6,328,712
U.S. Pat. No. 6,355,019
U.S. Pat. No. 6,365,338
U.S. Pat. No. 6,464,687
U.S. Pat. No. 6,484,721
U.S. Pat. No. 6,490,880
U.S. Pat. No. 6,492,103
U.S. Pat. No. 6,524,785
U.S. Pat. No. 6,552,083
U.S. Pat. No. 6,589,229
U.S. Pat. No. 6,626,329
U.S. Pat. No. 6,742,992
U.S. Pat. No. 6,764,472
U.S. Pat. No. 6,790,603
U.S. Pat. No. 6,800,096
U.S. Pat. No. 6,805,693
U.S. Pat. No. 6,908,448
U.S. Pat. No. 6,938,619
U.S. Pat. No. 6,958,154
U.S. Pat. No. 7,013,894
U.S. Pat. No. 7,022,107
U.S. Pat. No. 7,029,455
U.S. patent application Ser. No. 10/480,430
U.S. patent application Ser. No. 10/971,575
U.S. patent application Ser. No. 10/971,576
U.S. patent application Ser. No. 10/972,063
U.S. patent application Ser. No. 11/408,734
U.S. patent application Ser. No. 11/738,294
U.S. patent application Ser. No. 11/837,484
U.S. patent application Ser. No. 11/837,491
U.S. patent application Ser. No. 11/837,536
U.S. patent application Ser. No. 11/837,539
U.S. patent application Ser. No. 11/864,355
U.S. patent application Ser. No. 12/016,886
U.S. Patent Appln. 60/946,065
U.S. Patent Publn. 2005/0053912
U.S. Patent Publn. 2005/0136125
U.S. Patent Publn. 2005/0147692
U.S. Patent Publn. 2005/0170019
U.S. Patent Publn. 2007/0078113
U.S. Patent Publn. 2008/0085329
Alam, Antioxid Redox Signal, 4(4):559-62, 2002.
Amersi et al., Hepatology, 35(4):815-823, 2002.
Arisawa and Yamaguchi, J. Am. Chem. Soc. 125:6624-6625, 2003.
Austin-Ward and Villaseca, Revista Medica de Chile, 126(7):838-845, 1998.
Behringer et al., Crit. Care Med., 31(5):1523-1531, 2003.
Bellamy et al., Crit. Care Med., 24(2 Suppl):S24-47, 1996.
Bernard et al., J. Thorac. Cardiovasc. Surg. 90:235-242, 1985.
Bernard et al., N. Engl. J. Med., 346(8):557-563, 2002.
Blackstone et al., Science, 308:518, 2005.
Brizel, Seminars Radiation Oncol., 8(4Suppl):17-20, 1998.
Brouard et al., J. Biol. Chem., 277(20):17950-17961, 2002.
Bukowski et al., Clinical Cancer Res., 4(10):2337-2347, 1998.
Burns & Murphey, Arch. Biochem. Biophys., 339:33-39, 1997.1997
Burns et al., Arch. Biochem. Bipophys, 10:60-68, 1995.
Chivers, A Guide To Chalcogen-Nitrogen Chemistry, World Scientific Publ. Co. Pte. Ltd., Singapore, 2005.
Christodoulides et al., Microbiology, 144(Pt 11):3027-3037, 1998.
Clive et al., J. Org. Chem., 47:1641, 1982.
Cohen et al., Ann. Thorac. Surg., 67(5):1489-1491, 1999.
Curran, Seminars Radiation Oncol., 8(4Suppl):2-4, 1998.
Davidson et al., J. Immunother., 21(5):389-398, 1998.
Davis et al., In: Basic Methods in Molecular Biology, 2^{nd} Ed., Appleton and Lange (Eds.), Sec. 6-8, 1994.
Devillanova, Handbook of Chalcogen Chemistry: New Perspectives in Sulfur, Selenium and Tellurium, University of Cagliari, Italy, 2006.
Dhanasekaran et al., J. Biol. Chem., 279:37575-37587, 2004.
Dillman, Cancer Biother. Radiopharm., 14(1):5-10, 1999.
Dorman et al. Neurotoxicol. Teratol., 22(1):71-84, 2000.
Dulak et al., Antioxid. Redox Signal, 4(2):229-240, 2002.
Eto et al., Biochem. Biphys. Res. Commun., 293:1483-1488, 2002.
Evans et al., J. Amer. Chem. Soc., 112:4011-4030, 1990.
Ganther, Carcinogenesis 20:1657-66, 1999.
Gilbert et al., LANCET, 355:375-376, 2000.
Gorman et al., Toxicology, 187(1):25-38, 2003.
Guillemin et al., Cell, 89(1):9-12, 1997.
Guo and Zhang, J. Chem. Res. 2000:374-375, 2000.
Hanibuchi et al., Intl. J. Cancer, 78(4):480-45, 1998.
Hannan et al., JAMA, 290(6):773-780, 2003.
Hellstrand et al., Acta Oncologica, 37(4):347-353, 1998.
Higuchi and Fukamachi, Folia Pharmacologica Japonica, 73(3):307-319, 1977.
Hochachka et al., Comp. Biochem. Physiol. B Biochem. Mol. Biol., 130(4):435-459, 2001.
Hochachka et al., Proc. Natl. Acad. Sci. USA, 93(18):9493-94938, 1996.
Hui and Hashimoto, Infection Immun., 66(11):5329-5336, 1998.
Hyspler et al., J. Chromatography, 770:255-259, 2002.
Ju et al., J. Neuropathol. Exp. Neurol., 59(3):241-50, 2000.
Kamoun, Amino Acids 26, 243, 2004.
Kelso et al., J. Biol. Chem., 276:4588-4596, 2001.
Kilburn and Warshaw, Toxicology Indust. Health, 11(2):185-197, 1995.
Kilburn, Environ. Health, 54(3):150, 1999
Kilburn, Environ. Res., 81(2):92-99, 1999.
Kuroda et al., Transplantation, 46(3):457-460, 1988.
Ledingham et al., Circulation, 82(2):IV351-358, 1990.
Ledingham et al., J. Thorac. Cardiobasc. Surg., 93:240-246, 1987.
Lee et al., J. Nuc. Med. 26:72, 1985.
Lundgren-Eriksson et al., Anticancer Res. 2001 September-October; 21(5):3269-74
Mehlhorn et al., Cardiovasc Surg., 9(5):482-486, 2001.
Menasche et al., Eur. J. Cardio. Thorax. Surg., 8:207-213, 1994.
Michaels et al., Circulation, 106(23):e187-190, 2002.
Mitchell, In: Biological Interactions of Sulfur Compounds, Taylor & Francis, London, 1996.
Nystul et al., Science, 302(5647):1038-1041, 2003.
Otterbein et al., Am. J. Physiol. Lung Cell Mol. Physiol., 279(6):L1029-L1037, 2000.
Otterbein et al., Trends Immunol., 24(8):449-455, 2003.
Padilla et al., Molec. Biology of the Cell, 13:1473-1483, 2002.
Padilla et al., Proc. Natl. Acad. Sci. USA, 98(13):7331-7335, 2001.
Partlo et al., Neurotoxicology, 22(2):177-189, 2001.
PCT Appln. WO 94/17178
PCT Appln. WO 94/12285
PCT Appln. WO 94/14543
PCT Appln. WO 95/26234
PCT Appln. WO 95/26235
PCT Appln. WO 95/32807
Pietras et al., Oncogene, 17(17):2235-2249, 1998.
Pu et al., J. Amer. Chem. Soc., 56:1280-1283, 1991.
Qin et al., Proc. Natl. Acad. Sci. USA, 95(24):14411-14416, 1998.
Rager et al., NC Med. J., 65(1):18-25, 2004.
Reigan et al. J. Med. Chem., 48:392, 2005.
Remington's Pharmaceutical Sciences, 15^{th} ed., pp 1035-1038 and 1570-1580, Mack Publishing Company, Easton, Pa., 1980.
Rogers et al., Mamm. Genome, 8:711-713, 1997.
Ryter and Otterbein, BioEssays, 26:270-280, 2004.
Seburg abd Squires, Intl. J. Mass Spectrometry Ion Proc., 167/168:541, 1997.
Semenza, Cell, 98(3):281-284, 1999.
Semenza, Trends Mol. Med., 7(8):345-350, 2001.
Smith et al., Eur. J. Biochem., 263:709-716, 1999.
Stahl and Wermuth, eds., Handbook of Pharmaceutical Salts: Properties, Selection and Use, Verlag Helvetica Chimica Acta, 2002.
Steudel, Top. Curr. Chem., vol. 230, 2003a.
Steudel, Top. Curr. Chem., vol. 231, 2003b.
Struve et al., Neurotoxicology, 22(3):375-385, 2001.
Takano et al., Geochem. 1, 28:199-216, 1994.
Teodoro and O'Farrell, EMBO J., 22(3):580-587, 2003.
The Hypothermia After Cardiac Arrest Study Group et al., 2002.
Tisherman, Crit. Care Med., 32(2):S46-S50, 2004.
Van Voorhies et al., J. Exp. Biol., 203(Pt 16):2467-2478, 2000.
Viaravamurthy, Spectrochimica Acta Part A: Molecular and Biomolecular Spectroscopy, 54:2009-2017, 1998
Wang et al., Molec. Cell. Biol., 12(8):3399-3406, 1992.
Wang et al., Science, 259(5098):1173-1175, 1993.
Wang, Proc. Natl. Acad. Sci. USA, 93:8180-8184, 1994.
Williams et al., J. Amer. Chem. Soc., 113:9276-9286, 1991.
Williams, In: Synthesis of Optically Active α-Amino Acids, Pergamon Press, 1989.
Yaffe et al., Crit. Care Med., 32(2):S51-55, 2004.
Yaghi et al., Nature, 423(6941):705-714, 2003.
Ziegler, Ann. Rev. Biochem. 54, 305, 1985.

What is claimed is:

1. A polysulfide composition comprising (i) a peptide or a protein and (ii) a polychalcogenide salt, wherein the polychalcogenide salt is further defined as a compound of formula (I):
Z₂Sₙ (I)
wherein Z is a counterion and n is a number or average number ranging from 1-35, wherein the number or average number is an integer or non-integer ranging from 1-35.

2. The polysulfide composition of claim 1, wherein the peptide or the protein is a glutathione or albumin, respectively.

3. The polysulfide composition of claim 2, wherein the glutathione is oxidized glutathione or reduced glutathione.

4. The polysulfide composition of claim 1, wherein the counterion is an alkali metal ion.

5. The polysulfide composition of claim 1, wherein n represents an average number of 5.5.

6. A pharmaceutical composition comprising the polysulfide composition of claim 1 and a pharmaceutically acceptable carrier.

7. The pharmaceutical composition of claim 6, wherein the polysulfide composition is an aqueous solution.

8. A method of preparing an aqueous solution comprising the polysulfide composition of claim 1, comprising:
(a) mixing an aqueous solution comprising the peptide or the protein with
(b) a polychalcogenide salt, wherein the polychalcogenide salt is further defined as a compound of formula (I):
Z₂Sₙ (I)
wherein Z is a counterion and n is a number or average number ranging from 1-35, wherein the number or average number is an integer or non-integer ranging from 1-35.

9. The method of claim 8, wherein the polychalcogenide salt is further defined as a liquid solution formed from mixing two parts sodium sulfide nonahydrate with one part solid S₈.

10. The method of claim 8, wherein the peptide or the protein is a glutathione or albumin, respectively.

11. The method of claim 9, further comprising diluting the liquid solution formed from mixing two parts sodium sulfide nonahydrate with one part solid S₈ with HCl.

12. The method of claim 8, further defined as the following:
(a) mixing an aqueous solution comprising a mixture of albumin and a glutathione with
(b) a liquid solution formed by mixing two parts sodium sulfide nonahydrate with one part solid S₈.

13. The method of claim 12, wherein the ratio of albumin to total glutathione in (b) ranges from about 2:1 to about 3:1.

14. A method comprising providing the polysulfide composition of claim 1 to a subject, wherein the subject has or is at risk for a hypoxic/ischemic injury, trauma, hyperproliferative disease or condition, neurodegenerative disease, or transplant rejection.

15. The method of claim 14, wherein the hypoxic/ischemic injury, hemorrhagic shock, hyperproliferative disease or condition, neurodegenerative disease, inflammatory disease, transplant rejection, or autoimmune disease or condition is prevented or treated.

16. A method for preventing or treating damage in a subject comprising providing to the subject an effective amount of the polysulfide composition of claim 1.

17. The method of claim 16, wherein the damage is from trauma caused by an external physical source.

18. The method of claim 17, wherein the trauma is surgery.

19. The method of claim 16, wherein the subject has or is at risk for a hypoxic/ischemic injury, hyperproliferative disease or condition, neurodegenerative disease, or transplant rejection.

20. The method of claim 19, wherein the subject has or is at risk for hypoxic/ischemic injury.

21. The method of claim 20, wherein the hypoxic/ischemic injury is heart attack, cardiac surgery with cardio-pulmonary bypass, stroke, hematologic shock, or hemorrhagic shock.

22. The method of claim 20, wherein the injury involves hemorrhaging.

23. The method of claim 19, wherein the injury or disease is associated with a reduction in metabolism or temperature of the subject.

24. The method of claim 19, wherein the subject is provided with the polysulfide composition before, during, and/or after, or any combination thereof, the injury or onset or progression of the disease or condition.

25. The method of claim 24, wherein the subject is provided with the polysulfide composition in an amount and for a time that protects the subject from damage or death resulting from the injury or the onset or progression of the disease.

26. The method of claim 16, wherein the subject is provided with the polysulfide composition under hypoxic or anoxic conditions or prior to exposure to hypoxic or anoxic conditions.

27. The method of claim 16, wherein the polysulfide composition is provided to the subject as comprised in or as a liquid.

28. The method of claim 16, wherein the subject is provided the polysulfide composition for a period of time between about 30 seconds and 30 days.

29. The method of claim 16, wherein the subject is provided the polysulfide composition by administration to the subject intravenously, intradermally, intraarterially, intraperitoneally, intralesionally, intracranially, intraarticularly, intraprostaticaly, intrapleurally, intratracheally, intranasally, intrathecally, intravitreally, intravaginally, intrarectally, topically, intratumorally, intramuscularly, intraperitoneally, intraocularly, subcutaneously, subconjunctival, intravesicularlly, mucosally, intrapericardially, intraumbilically, intraocularally, orally, topically, locally, by inhalation, by injection, by infusion, by continuous infusion, by localized perfusion, via a catheter, or via a lavage.

30. The method of claim 16, wherein the subject is provided the polysulfide composition through infusion, inhalation, injection, catheterization, immersion, lavage, perfusion, topical application, absorption, adsorption, or oral administration.

31. The method of claim 16, further comprising exposing the subject to a controlled temperature and/or pressure environment.

32. The method of claim 16, further comprising identifying a subject in need of treatment.

33. The method of claim 16, further comprising evaluating whether the subject is metabolizing the polysulfide composition.

34. A method for treating a subject with a blood substitute comprising providing to the subject an effective amount of the polysulfide composition of claim 1.

35. A method of protecting a mammal from suffering cellular damage from a disease or adverse medical condition, comprising providing to the mammal an effective amount of the polysulfide composition of claim 1 prior to the onset or progression of the disease or condition.

36. The method of claim 35, wherein the disease or adverse medical condition is selected from the group consisting of: hemorrhagic shock, myocardial infarction, acute coronary syndrome, cardiac arrest, neonatal hypoxia/ischemia, ischemic reperfusion injury, unstable angina, post-angioplasty, aneurysm, trauma, and blood loss.

37. A method for treating hemorrhagic shock in a patient comprising providing to the patient an effective amount of the polysulfide composition of claim 1.

38. The method of claim 37, wherein the compound does not affect blood pressure by more than 10 mm Hg.

39. A method of enhancing survivability in a subject comprising providing to the subject an effective amount of the polysulfide composition of claim 1.

40. The method of claim 39, wherein the subject is suffering from or susceptible to suffering from a disease or adverse medical condition.

41. The method of claim 40, wherein the disease is thalassemia, sickle cell disease, or cystic fibrosis.
